(19) United States
(12) Patent Application Publication
(10) Pub. No.: US 2025/0090588 A1
(43) Pub. Date: Mar. 20, 2025

(54) METHODS OF TREATING OR PREVENTING PREMATURE OVARIAN INSUFFICIENCY, POLYCYSTIC OVARY SYNDROME, OR INFERTILITY USING EXOSOMES OR MESENCHYMAL STEM CELLS

(71) Applicant: The University of Chicago, Chicago, IL (US)

(72) Inventors: Ayman Al-Hendy, Hinsdale, IL (US); Hang-Soo Park, Chicago, IL (US)

(73) Assignee: The University of Chicago, Chicago, IL (US)

(21) Appl. No.: 18/577,180

(22) PCT Filed: Jul. 6, 2022
(86) PCT No.: PCT/US2022/073467

§ 371 Date: Jan. 5, 2024

Related U.S. Application Data
(60) Provisional application No. 63/218,793, filed on Jul. 6, 2021.
(60) Provisional application No. 63/236,658, filed on Aug. 24, 2021.

(51) Int. Cl. A61K 35/28; A61K 38/18; A61K 38/20; A61P 15/00; C12N 5/071; C12N 5/0775; C12N 15/113

(52) U.S. Cl. CPC A61K 35/28; A61K 38/1841; A61K 38/1875; A61K 38/2066; A61P 15/00; C12N 5/0663; C12N 5/0682; C12N 15/113; C12N 2310/141; C12N 2502/1358

(57) ABSTRACT

In aspects, the present disclosure provides a method of treating or preventing premature ovarian insufficiency or polycystic ovary syndrome in a female mammal, the method comprising, consisting essentially of, or consisting of administering to the female mammal an effective amount of (i) mesenchymal stem cells (MSCs) or secretome from MSCs, wherein the MSCs overexpress (a) miR144, (b) BMP-2, (c) TGFβ1, (d) IL-10, or (e) any combination of (a), (b), (c) and (d); (ii) exosomes produced by MSCs, wherein the MSCs overexpress (a) miR144, (b) BMP-2, (c) TGFβ1, (d) IL-10, or (e) any combination of (a), (b), (c) and (d); and/or (iii) exosomes comprising one or more effectors, wherein the one or more effectors comprises, consists essentially of, or consists of (a) miR144, (b) BMP-2, (c) TGFβ1, (d) IL-10, or (e) any combination of (a), (b), (c) and (d), and wherein the amount of the effector per exosome is greater than the amount of the effector per exosome when produced by unmodified MSCs. In aspects, the present disclosure provides a method of preparing MSCs, exosomes, and secretome. In aspects, the present disclosure provides a method of preventing chemotherapy-induced damage in a male mammal.

### CROSS-REFERENCE TO RELATED APPLICATIONS

This patent application claims the benefit of co-pending U.S. Provisional Patent Application No. 63/218,793, filed Jul. 6, 2021, and co-pending U.S. Provisional Patent Application No. 63/236,658, filed Aug. 24, 2021, each of which is incorporated by reference herein in its entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 51,130 Byte XML file named “763230.xml” created on Jul. 1, 2022.

### BACKGROUND

Premature ovarian insufficiency (POI) is characterized by hypoestrogenism, amenorrhea, elevated gonadotropin levels, and reduced follicle counts, along with infertility in women under the age of 40 years. POI is frequently caused by chemotherapy in cancer patients due to gonadotoxic chemotherapy reagents damaging granulosa cells, which are essential for follicular development. Therefore, many women who had a cancer at a young age may have later infertility issues even if they survive the cancer. POI has also been referred to as premature ovarian insufficiency (POF).

Hyperandrogenism, ovulatory dysfunction, and polycystic ovarian morphology are key features of polycystic ovary syndrome (PCOS), leading to excessive inflammation and increased androgen production from ovarian theca cells. PCOS is the most common endocrine and metabolic disorder in reproductive-age women. Around 15-18% of reproductive-age women suffer from PCOS. Many women with PCOS also have metabolic aberrations such as insulin resistance, dyslipidemia, and hypertension, which could be considered as a cardiovascular risk factor.

There is an ongoing need in the art to treat or prevent POI, PCOS, and infertility in general.

### BRIEF SUMMARY

In aspects, the present disclosure provides a method of treating or preventing POI or PCOS in a female mammal, the method comprising, consisting essentially of, or consisting of administering to the female mammal an effective amount of mesenchymal stem cells (MSCs) or secretome from MSCs, wherein the MSCs overexpress (a) miR144, (b) BMP-2, (c) TGFβ1, (d) IL-10, or (e) any combination of (a), (b), (c) and (d).

In aspects, the present disclosure provides a method of treating or preventing POI or COS) in a female mammal, the method comprising, consisting essentially of, or consisting of administering to the female mammal an effective amount of exosomes produced by MSCs, wherein the MSCs overexpress (a) miR144, (b) BMP-2, (c) TGFβ1, (d) IL-10, or (e) any combination of (a), (b), (c) and (d).

In aspects, the present disclosure provides a method of treating or preventing POI or PCOS in a female mammal, the method comprising, consisting essentially of, or consisting of administering to the female mammal an effective amount of exosomes comprising one or more effectors, wherein the one or more effectors comprises, consists essentially of, or consists of (a) miR144, (b) BMP-2, (c) TGFβ1, (d) IL-10, or (e) any combination of (a), (b), (c) and (d), and wherein the amount of the effector per exosome is greater than the amount of the effector per exosome when produced by unmodified MSCs.

In aspects, the present disclosure provides a method of preparing mesenchymal stem cells (MSCs) for treating or preventing premature ovarian insufficiency (POI) or polycystic ovary syndrome (PCOS) in a female mammal, the method comprising collecting MSCs in an amount of about 4×10⁷ for administration in a single dosage.

In aspects, the present disclosure provides a method of preparing ovarian tissue-specific exosomes and/or secretome, the method comprising co-culturing human ovarian granulosa cells (hGrC1) with mesenchymal stem cells (MSCs).

In aspects, the present disclosure provides a method of preventing chemotherapy-induced damage in a male mammal, the method comprising administering to the mammal an effective amount of exosomes and/or secretome prepared from mesenchymal stem cells (MSCs).

Additional aspects of the present disclosure are as described herein.

### BRIEF DESCRIPTION OF THE DRAWINGS

FIGS. 1A-1O are bar graphs and gel sections that show the effect of BM-hMSC secretome on H295R cells and human PCOS theca cells. After 24 hours of treatment, (FIG. 1A) percentage of Ki67 positive cells, (FIG. 1B) percentage of Annexin V positive cells, and (FIG. 1C) percentage of Annexin V+/7AAD positive cells in the BM-hMSC secretome-treated vs. control (basal media) H295R cells. (FIG. 1D) Relative mRNA expression of CYP17A1, (FIG. 1E) CYP11A1, and (FIG. 1F) DENND1A in BM-hMSC secretome-treated vs. control H295R cells. (FIG. 1G) CYP17A1 protein expression, (FIG. 1H) CYP11A1 protein expression, and (FIG. 11) DENND1A protein expression in BM-hMSC secretome-treated vs. control H295R cells. (FIG. 1J) Relative mRNA expression of CYP17A1 in human PCOS theca cells (n=3) after BM-hMSC secretome treatment vs. control (basal media). (FIG. 1K) Average protein expression level of CYP17A1 in human PCOS theca cells (n=2) after BM-hMSC secretome treatment vs. control. (FIG. 1L) Testosterone secretion by H295R cells and (FIG. 1M) human PCOS theca cells; BM-hMSC secretome vs. control group. (FIG. 1N) Relative gene expression of inflammatory marker IL1B and (FIG. 1O) TNFA in BM-hMSC secretome-treated vs. control basal media in H295R cells. *: p<0.05, **: p<0.005, ***: p<0.0005; NS: Not significant.

FIGS. 2A-2I are bar graphs that show the effect of IL-10 on H295R cells. (FIG. 2A) Concentration of IL-10 secreted by BM-hMSC. (FIG. 2B) Relative gene expression of CYP17A1, (FIG. 2C) CYP11A1, and (FIG. 2D) DENND1A after IL-10 treatment. (FIG. 2E) Testosterone secretion and (FIG. 2F) androstenedione secretion by H295R cells after IL-10 treatment. (FIG. 2G) Relative gene expression of inflammatory markers IL-6 (IL6), (FIG. 2H) TNF-α (TNFA), and (FIG. 2I) IL-10 (IL1B) after IL-10 treatment. *: p<0.05, **: p<0.005; NS: Not significant.

FIGS. 3A-3N are graphs and pictures that show BM-hMSC injection into the ovary reverses metabolic phenotypes in the LTZ-induced PCOS mouse model. (FIG. 3A) Effect of LTZ on body weight in LTZ-treated mice (PCOS) and matched controls. Mean body weight after week 5 was significantly higher in the PCOS group compared with matched controls. (FIG. 3B) Percent rate of increase in body weight in control mice and PCOS mice. (FIG. 3C) Glucose tolerance test was performed on starved mice and mice after intra-peritoneal (i.p.) glucose injection, monitored at the indicated time points for blood glucose level represented in mg/dL. (FIG. 3D) Blood glucose level at time 0 min after 16 h overnight fast. (FIG. 3E) Comparison of blood glucose level 2 hours after glucose i.p. injection. BM-hMSC enhances energy expenditure in the PCOS mouse model. (FIG. 3F) Oxygen (O₂), (FIG. 3G) carbon dioxide (CO₂), (FIG. 3H) respiratory exchange ratio (RER), and (FIG. 3I) heat production presented in histograms comparing energy expenditure profiles of the PCOS group and BM-hMSC-treated PCOS group. (FIGS. 3J-3N) BM-hMSC induces browning of white fat in the PCOS mouse model. (FIG. 3J) UCP-1 immunohistochemistry staining of white gonadal fat. Scale bar is 50 μm. (FIG. 3K) Relative gene expression of UCP-1 (Ucp1), (FIG. 3L) PGC-1α (Pgc1a), (FIG. 3M) Cidea, and (FIG. 3N) Prdm16 in white fat from PCOS mice and BM-hMSC-treated PCOS mice. *: p<0.05, **: p<0.005.

FIGS. 4A-4H are graphs and pictures that show BM-hMSC injection into the ovary restores fertility in LTZ-induced PCOS mouse model. (FIG. 4A) Morphology of ovary from a normal mouse (control), LTZ-induced PCOS mouse (PCOS), and BM-hMSC-treated PCOS mouse (BM-hMSC). Scale bar is 500 μm. (FIG. 4B) Pregnancy rate of the control group (8 out of 10), PCOS group (1 out of 10), and BM-hMSC-treated group (8 out of 10). (FIG. 4C) The mating index (Mating rate) of the control group, PCOS group, and BM-hMSC-treated group. Morphology of pups and implantation site in the uterus of (FIG. 4D) control group, (FIG. 4E) PCOS group, and (FIG. 4F) BM-hMSC-treated group. (FIG. 4G) Average number of pups from the control group, PCOS group, and BM-hMSC group. (FIG. 4H) Average body weight of pups post-natal day 10. *: p<0.05, ***: p<0.0005; NS: Not significant.

FIGS. 5A-5G are bar graphs and gel sections that show BM-hMSC injection into the ovary reverses altered gene expression in ovarian tissue of LTZ-induced PCOS mice. (FIG. 5A) Relative gene expression of Cyp17a1 (FIG. 5B) Cyp19a1, (FIG. 5C) Fshr, and (FIG. 5D) Vegfa in the ovary from control, untreated PCOS, and BM-hMSC-treated PCOS mice. (FIG. 5E) Immunoblot of CYP17A1 and VEGFA in the ovary. (FIG. 5F) Quantification of CYP17A1 and (FIG. 5G) VEGFA in the ovary from control, PCOS, and BM-hMSC-treated PCOS mice. *: p<0.05, **: p<0.005, ***: p<0.0005; NS: Not significant.

FIGS. 6A-6K are graphs and assays that show the effect of BM-hMSC on cytokines in the LTZ-induced PCOS mouse model. (FIG. 6A) Relative gene expression of IL-10 (Il10) and (FIG. 6B) IL-10R (Il10r) in the ovary from control, untreated PCOS, and BM-hMSC-treated PCOS mice. (FIG. 6C) Serum levels of inflammatory regulatory markers by antibody-based membrane assay. (FIG. 6D) Fold change of serum IL-10 (FIG. 6E) INF-γ and (FIG. 6F) TIMP-2 in control, PCOS, and BM-hMSC-treated PCOS mice. (FIGS. 6G-6J) Pro-inflammation marker mRNA expression in adipose tissue. (FIG. 6G) Relative gene expression of IL-6 (Il6) (FIG. 6H) IL-10 (Il1b), (FIG. 6I) CCL2 (Ccl2), and (FIG. 6J) CD11c (Cd11c) in untreated and BM-hMSC treated PCOS mice. (FIG. 6K) Schematic of the proposed model for BM-hMSC therapeutic effect in PCOS. A positive stimulation loop between inflammation, androgen production, and metabolic abnormalities could lead to PCOS. BM-hMSC alleviate the inflammation via secretion of anti-inflammatory factor IL-10, which suppresses androgen secretion by ovarian theca cells. Those effects in turn can improve the metabolic abnormalities. Regulation of inflammation through IL-10 leads to improved fertility in PCOS. Illustration was created with BioRender.com. *: p<0.05, **: p<0.005; NS: Not significant.

FIGS. 7A-7D are graphs that show human bone marrow mesenchymal stem cells (BM-hMSC) characterization. BM-hMSC were isolated from a 32-year-old healthy adult female bone marrow (non-diabetic). Cells were characterized by (FIG. 7A) lineage negative for CD11b, CD34, CD19, CD45, and HLA-DR, and lineage positive for (FIG. 7B) CD90, (FIG. 7C) CD73, and (FIG. 7D) CD105.

FIGS. 8A-8F are graphs and pictures that show BM-hMSC reverse brown fat tissue phenotype in the LTZ-induced PCOS mouse. Immunohistochemistry of brown fat tissue by (FIG. 8A) PD-L1 and (FIG. 8B) UCP-1. (FIG. 8C) Relative gene expression of UCP-1 in brown fat of PCOS mice (1.0±0.03 fold) and BM-hMSC-treated PCOS mice (4.66±0.07 fold). (FIG. 8D) Relative gene expression of PGC-1α in brown fat of PCOS mice (1.0±0.10 fold) and BM-hMSC-treated PCOS mice (2.38±0.09 fold). (FIG. 8E) Relative gene expression of Cidea in brown fat of PCOS mice (1.0±0.04 fold) and BM-hMSC-treated PCOS mice (2.31±0.08 fold). (FIG. 8F) Relative gene expression of PRDM16 in the brown fat of PCOS mice (1.0±0.12 fold) and BM-hMSC-treated PCOS mice (5.19±0.19 fold). Data presented as the mean±SD. *: p<0.05, **: p<0.005.

FIGS. 9A-9H are graphs and pictures that show BM-hMSC reverse adipose tissue adipokines in LTZ-induced PCOS mouse model. (FIG. 9A) H&E staining of white gonadal fat, arrows indicate immune cells infiltrating adipocyte interstitial space in PCOS mice treated with BM-hMSC and untreated PCOS mice. (FIG. 9B) The average size of adipocytes in PCOS group (2172.7±610.5 μm²) and BM-hMSC-treated PCOS group (828.7±223.1 μm²). (FIGS. 9C-9H) Real-time PCR of brown and white gonadal fat comparing untreated PCOS group and BM-hMSC-treated PCOS group. (FIG. 9C) Relative gene expression of adiponectin in brown fat of PCOS mice (1.0±0.13 fold) and BM-hMSC-treated PCOS mice (15.75±0.12 fold). (FIG. 9D) Relative gene expression of leptin in brown fat of PCOS mice (1.0±0.01 fold) and BM-hMSC-treated PCOS mice (0.16±0.01 fold). (FIG. 9E) Ratio of leptin to adiponectin in the brown fat of PCOS mice (1.0±0.13 fold) and BM-hMSC-treated mice (0.01±0.01 fold). (FIG. 9F) Relative gene expression of adiponectin in white fat of PCOS mice (1.0±0.14 fold) and BM-hMSC-treated mice (6.00±0.57 fold). (FIG. 9G) Relative gene expression of adiponectin in white fat of PCOS mice (1.0±0.20 fold) and BM-hMSC-treated mice (0.07±0.02 fold). (FIG. 9H) Ratio of leptin to adiponectin in white fat of PCOS mice (1.0±0.15 fold) and BM-hMSC-treated PCOS mice (0.4±0.08 fold). Data presented as the mean±SD. *: p<0.05, **: p<0.005.

FIGS. 10A-10D are bar graphs that show hormonal analysis in the LTZ-induced PCOS mouse model. Average serum hormone levels (n=8/group). (FIG. 10A) Level of serum testosterone (T) in control (31.8±9.5 ng/dL), PCOS (47.3±11.4 ng/dL), and BM-hMSC-treated PCOS groups (48.8±8.4 ng/dL). (FIG. 10B) Level of serum estradiol (E2) in control (7.6±1.4 pg/dL), PCOS (6.8±1.2 pg/dL), and BM-hMSC-treated PCOS groups (6.9±0.63 pg/dL). (FIG. 10C) Level of serum luteinizing hormone (LH) in control (0.7±0.58 ng/dL), PCOS (0.1±0.03 ng/dL), and BM-hMSC-treated PCOS groups (0.3±0.13 ng/dL). (FIG. 10D) Level of serum follicle-stimulating hormone (FSH) in control (6.8±0.6 ng/dL), PCOS (4.6±1.1 ng/dL), and BM-hMSC-treated PCOS groups (7.3±1.5 ng/dL). Data presented as the mean±SD. *: p<0.05; NS: Not significant.

FIGS. 11A-11I are pictures and graphs that show the effect of BM-hMSC on the endometrium in the LTZ-induced PCOS mouse model. (FIG. 11A) Morphology of mouse endometrium tissue in control, PCOS, and BM-hMSC-treated PCOS mice (H&E staining). (FIG. 11B) Comparison of relative AIB1 gene expression level in control (1.00±0.15 fold), PCOS (2.08±0.29 fold), BM-hMSC-treated PCOS (1.04±0.30 fold) mouse endometrium. (FIG. 11C) Comparison of relative AR gene expression level in control (1.08±0.54 fold), PCOS (1.44±0.31 fold), and BM-hMSC-treated PCOS (1.05±0.12 fold) mouse endometrium. (FIG. 11D) Comparison of relative ERP gene expression level in control (1.03±0.35 fold), PCOS (1.50±0.63 fold), and BM-hMSC-treated PCOS (1.01±0.68 fold) mouse endometrium. (FIG. 11E) Comparison of relative Ki67 gene expression level in control (1.08±0.47 fold), PCOS (0.26±0.07 fold), and BM-hMSC-treated PCOS (0.57±0.09 fold) mouse endometrium. (FIG. 11F) Comparison of relative IL6 gene expression level in control (1.02±0.24 fold), PCOS (2.26±0.44 fold), and BM-hMSC-treated PCOS (1.13±0.24 fold) mice endometrium. (FIG. 11G) Comparison of relative IL16 gene expression level in control (1.00±0.04 fold), PCOS (1.91±0.14 fold), and BM-hMSC-treated PCOS (0.83±0.09 fold) mouse endometrium. (FIG. 11H) Comparison of relative CCL2 gene expression level in control (1.00±0.04 fold), PCOS (1.46±0.12 fold), and BM-hMSC-treated PCOS (0.70±0.19 fold) mouse endometrium. (FIG. 11I) Comparison of relative TNFα gene expression level in control (1.00±0.12 fold), PCOS (2.05±0.16 fold), and BM-hMSC-treated PCOS (0.97±0.29 fold) mouse endometrium. Data presented as the mean±SD. *: p<0.05, **: p<0.005, ***: p<0.0005; NS: Not significant.

FIGS. 12A-12E are pictures and graphs that show the effect of BM-hMSC secretome injection on the ovary, white fat, brown fat, and fertility in the LTZ-induced PCOS mouse model. (FIG. 12A) Morphology of ovary with H&E staining in control mice (Control), PCOS mice (PCOS), and BM-hMSC secretome-treated PCOS mice (Secretome). (FIG. 12B) Size of adipocyte in white fat tissue from the control group (926±266 μm2), PCOS group (2173±610 μm2), and Secretome group (829±223 μm2). (FIG. 12C) Density of brown fat analyzed with UCP1 by immunohistochemistry. (FIG. 12D) Pregnancy rate of the control group (4 out of 4), the PCOS group (1 out of 4), and the Secretome group (4 out of 4). (FIG. 12E) Average number of pups from the control group (7.6±1.5), PCOS group (1.3±2.3), and the Secretome group (9.0±2.2). Data presented as the mean±SD. *: p<0.05.

FIGS. 13A-13D are bar graphs that show the effect of BMP-2 on H295R cells. The concentration of BMP-2 was essentially that same in MSC isolated after different culture passages (FIG. 13A). Treatment with human recombinant BMP-2 significantly decreased H295R cell proliferation in a dose- and time-dependent manner (FIGS. 13B-D). RT-PCR results (FIGS. 13E-G) show that BMP-2 treatment significantly downregulated the expression of two key androgen-synthesizing enzymes, CYP17A1 and DENND1A, but had no significant effect on CYP11A1 gene expression. The effect of BMP-2 on the expression of estrogen-producing genes Cyp19 and StAR was analyzed in human ovarian granulosa cells (hGrC1), a model of POI (FIGS. 13H-I). The gene expression levels were not consistent for the dose of BMP2. Only the specific BMP-2 concentration 25 ng/ml showed significant enhancement of Cyp19 and StAR gene expression. N/S: P>0.05, *: P≤0.05, **: P≤0.01, ***: P≤0.001, ****: P≤0.0001.

FIGS. 14A-14E are bar graphs that show the effect of TGFβ-1 on H295R cells. Initially, the concentration of TGFβ-1 was measured in unmodified MSC secretomes grown in human dermal fibroblast conditioned medium (HDF CM), showing the level of TGFβ-1 increased with time, (FIG. 14A). Then, RT-PCR results show that TGFβ-1 treatment significantly downregulated the expression of two key androgen-synthesizing enzymes, CYP17A1 and DENND1A, but had no significant effect on CYP11A1 gene expression (FIGS. 14B-D). TGF β-1 treatment also significantly suppressed testosterone level in androgen-producing H295R cells in conditioned media, an in vitro model of PCOS (FIG. 14E). N/S: P>0.05 *: P≤0.05, **: P≤0.01 ***: P≤0.001, ****: P 0.0001.

FIGS. 15A-15D are bar graphs that show the effect of human mesenchymal bone marrow stem cells overexpressing miR144p on HGrC1 cells. After treating cyclophosphamide-treated HGrC1 with condition media for 24 hours, cell proliferation and steroidogenesis gene expression were analyzed. Cell proliferation and expression of steroidogenesis genes (CYP19A1 abd StAR) was significantly higher after treatment with miR144 overexpressing-BM-HSCs, compared to unenhanced BM-MSCs condition media (CM) (FIG. 15A). Then three different clones of BM-HSCs overexpressing miR144 were developed. Clones #2 and #3 stimulated the proliferation of hGrC1 cells (FIG. 15B), as well as enhanced the gene expression of estrogen producing-gene Cyp19a1 in CM treated-hGrC1 (FIG. 15C). All three clones enhanced the gene expression of estrogen producing gene StAR (Steroidogenic Acute Regulatory protein-encoding gene) in CM treated hGrC1 cells (FIG. 15D). N/S: P>0.05, *: P≤0.05, **: P≤0.01, ***: P≤0.001, ****: P≤0.0001.

FIGS. 16A-16C are images and bar graphs showing generation of enhanced MSCs which overexpress miR144. MSCs were induced for overexpression of miR144 by lentivirus transfection and labeled with GFP for isolation. miR144 overexpressing GFP positive MSCs were purified using a cell sorter and used for further analysis (FIG. 16A). GFP positive cells were confirmed after lentivirus transfection (FIG. 16B). Using a cell sorter, the GFP positive population was purified (99.89%), and a high expression level of miR144 was confirmed in the purified MSC population (FIG. 16C). N/S: P>0.05, *: P≤0.05, **: P≤0.01, ***: P≤0.001. ****: P≤0.0001.

FIGS. 17A-17D are dot plots showing characterization of miR144 overexpressing MSCs. miR144-overexpressing MSCs express the positive markers of MSC such as CD90, CD105, CD73 and are negative for the negative markers (multi-PE, CD45, CD34, CD11b, CD19 and HLA-DR) (FIG. 17A). miR144-overexpressing MSCs still have differentiation potential such as osteogenic potential (FIG. 17B), adipogenic potential (FIG. 17C), and chondrogenic potential (FIG. 17D).

FIGS. 18A and 18B are images and bar graphs showing characterization of miR144-overexpressing MSC-derived exosomes. For this characterization, exosomes were purified by a standard isolation protocol. The size and morphology were confirmed by transmission electron microscopy (TEM), and exosome-specific marker protein expression was analyzed by western blot (FIG. 18A). The size and concentration of miR144-overexpressing exosomes were analyzed by Nanoparticle Tracking Analysis (NTA) (FIG. 18B). N/S: P>0.05, *: P≤0.05, **: P≤0.01, ***: P≤0.001, ****: P≤0.0001.

FIGS. 19A-19C are images and bar graphs showing the therapeutic potential of secretion from MSC which was produced in a co-culture system. The data shows the effect of secretome treatment on hGrC1 proliferation and viability after chemotherapy. Healthy hGrC1 cells (H-GCs) were treated with 200 μg/ml cyclophosphamide to make sick hGrC1 (S-GCs). Twenty-four hours after secretome therapy, cell viability (FIG. 19A) and cell number (FIG. 19B) successfully increased in S-GC treated by secretome purified from MSC/S-GS co-culture and MSC/H-GS co-culture. Secretome purified from MSC/S-GS co-culture and MSC/H-GS co-culture also increases the levels of expression Ki67, cdk1, and CCNB1 (FIG. 19C) in S-GC. N/S: P>0.05, *: P≤0.05, **: P≤0.01, ***: P≤0.001, ****: P≤0.0001.

FIGS. 20A and 20B are images and bar graphs showing the anti-apoptotic potential of enhanced MSC secretome produced in a co-culture system. Healthy hGrC1 (H-GCs) were treated with 200 μg/ml cyclophosphamide to make sick hGrC1 (S-GCs). The S-GC were treated by MSC secretome, MSC/S-GC co-culture secretome, and MSC/H-GC co-culture secretome for 24 hours. MSC/S-GC co-culture secretome and MSC/H-GC co-culture secretome therapy significantly increased the Akt protein expression (anti-apoptotic protein) as significantly reduced Bax protein expression (proapoptotic protein) in sick GC (FIG. 20A). MSC/S-GC co-culture secretome and MSC/H-GC co-culture secretome therapy increased the expression of Akt and Bcl2 genes (anti-apoptotic protein) as well as significantly decreased the expression of Bax, GADD, and Casp3 (proapoptotic protein) in S-GC (FIG. 20B). N/S: P>0.05 *: P≤0.05, **: P≤0.01 ***: P≤0.001, ****: P≤0.0001.

FIGS. 21A and 21B are bar graphs showing the effect of enhanced MSC secretome produced in a co-culture system on steroidogenesis marker expression. Healthy hGrC1 (H-GCs) were treated with 200 μg/ml cyclophosphamide to make sick hGrC1 (S-GCs). The S-GC were treated by MSC secretome, MSC/S-GC co-culture secretome, and MSC/H-GC co-culture secretome for 24 hours. The AMH levels were significantly increased in all three treated groups compared to control (FIG. 21A). The effect of MSC secretome, MSC/S-GC co-culture secretome, and MSC/H-GC co-culture secretome treatment on hGrC1 marker genes was evaluated by gene expression analysis of estrogen-producing genes Cyp19A1, StAR, and FSHR. The expression of StAR and FSHR increased in S-GC treated by MSC/H-GC co-culture secretome, and the expression of estrogen-producing genes Cyp19A1 significantly increased in S-GC treated by MSC/H-GC co-culture secretome (FIG. 21B). N/S: P>0.05, *: P≤0.05, **: P≤0.01, ***: P≤0.001, ****: P≤0.0001.

FIGS. 22A-22F are images and bar graphs showing the protective effect on mouse fertility of exosome treatment before chemotherapy. Before chemotherapy (CTX), exosome treatment (1×10⁸ particle/ovary) was performed through intra-ovarian injection (FIG. 22A). The treated mice were still fertile, while untreated mice became infertile (FIG. 22B showing average AMH level, FIG. 22C showing pregnancy rate, and FIG. 22D showing number of pups). Delivered offspring from exosome-treated mice grew well compared with pups from healthy mice (FIG. 22E showing average body weight and FIG. 22F showing images). N/S: P>0.05, *: P≤0.05, **: P≤0.01, ***: P≤0.001, ****: P≤0.0001.

### DETAILED DESCRIPTION

In aspects, the present disclosure provides a method of treating or preventing POI or PCOS in a female mammal, the method comprising, consisting essentially of, or consisting of administering to the female mammal an effective amount of MSCs or secretome from MSCs, wherein the MSCs overexpress (a) miR144, (b) BMP-2, (c) TGFβ1, (d) IL-10, or (e) any combination of (a), (b), (c) and (d).

MSCs are adult stem cells that can be isolated from a variety of tissues, including bone marrow, adipose, umbilical cord tissue, or blood. MSCs are multipotent, meaning they can differentiate into many types of mature cells, such as adipogenic, osteogenic, and chondrogenic cells. MSCs characteristically express surface markers for CD90, CD73, and CD105, while lacking expression of CD34, CD11b, CD19, CD45, and HLA-DR surface antigens. In aspects, the MSCs can be placenta, bone marrow, umbilical cord MSCs, or any combination thereof.

The secretome of MSCs contains substances released by MSCs into the medium, milieu, or environment surrounding the MSCs. Secretome includes factors that are secreted by a cell to an extracellular space, which can be the components of machineries for protein secretion and the native secreted proteins. The MSC secretome can contain various MSC-derived factors such as exosomal miR144, BMP-2, TGFβ1, and IL-10.

MicroRNA-144 (miR-144) comprises a family of microRNA precursors of about 22 nucleotides found in mammals, including humans. miRNA-144 has been reported to be downregulated in the plasma of POF (POI) patients. miR-144-5p can be derived from BM MSC exosomes. The sequence of miR144-5p is 5′-GGA UAU CAU CAU AUA CUG UAA G-3′ (SEQ ID NO: 1).

Bone morphogenetic proteins (BMPs) are among the many growth factors secreted by BM-hMSCs; these proteins play a key role in female fertility and are involved in all stages of folliculogenesis. BMPs are multifunctional growth factors that belong to the transforming growth factor β (TGFβ) superfamily. Studies have suggested that BMPs may play an important role in the regulation of PCOS-related characteristics.

The anti-inflammatory factor IL-10 is produced and secreted by BM-hMSC. Chronic low-grade inflammation has been implicated as a driver of pathophysiology in PCOS, and it has been reported that interventions involving BM-hMSC or its secreted factors can improve the endocrine and metabolic abnormalities observed in PCOS, as well as fertility outcomes.

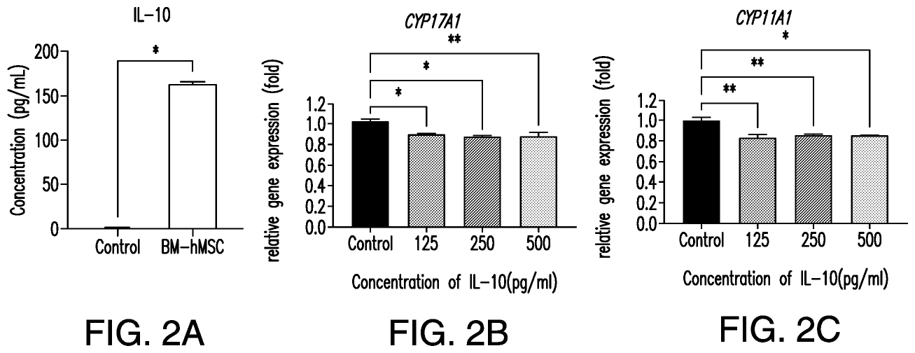

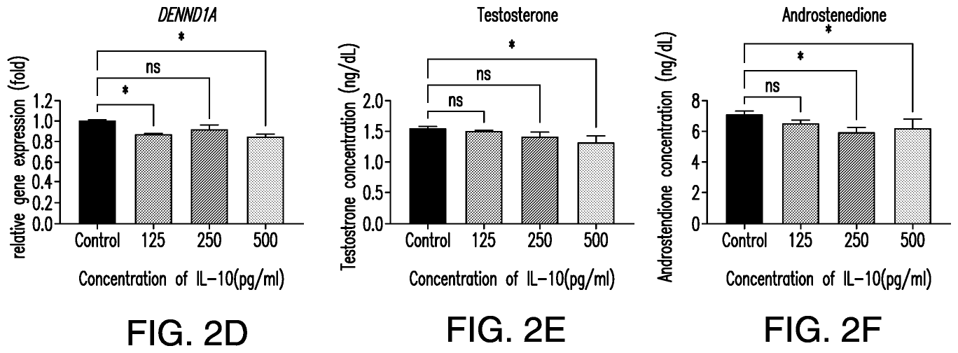

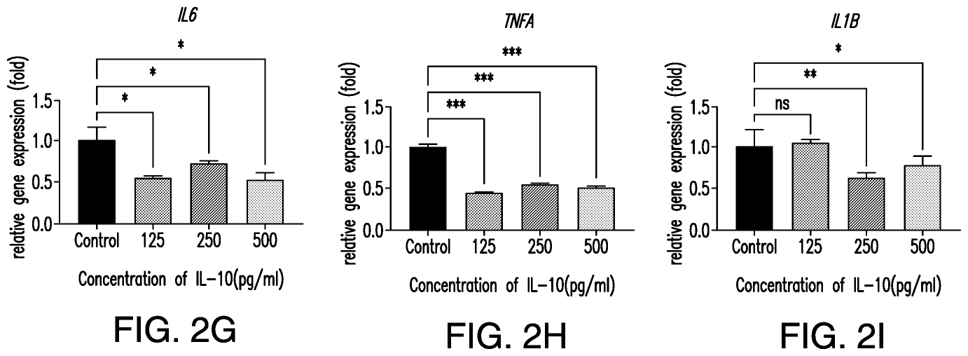

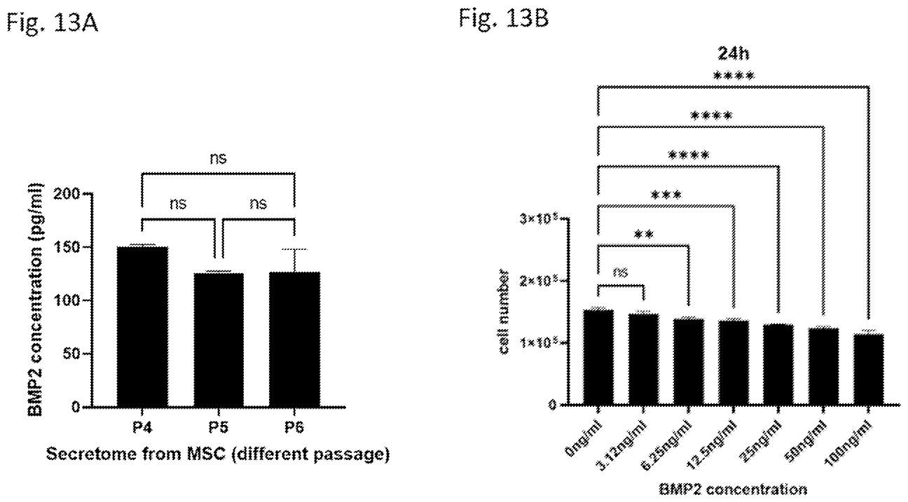

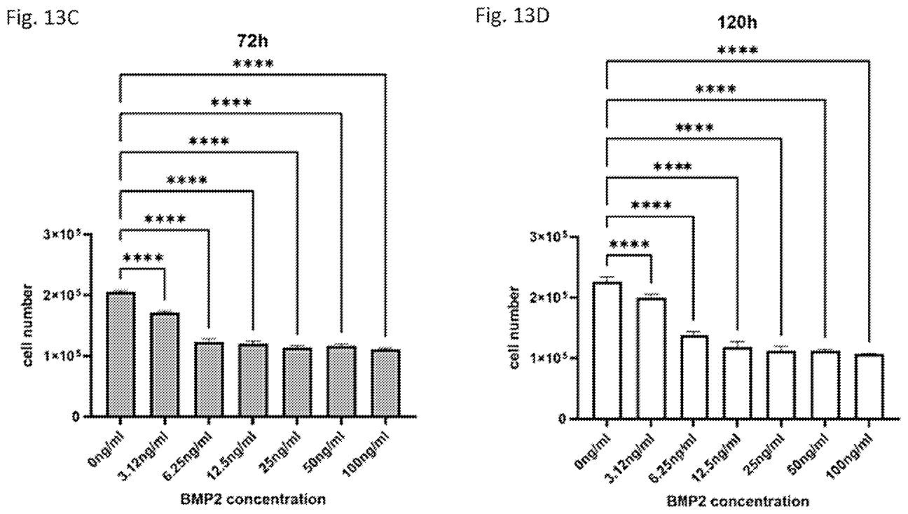

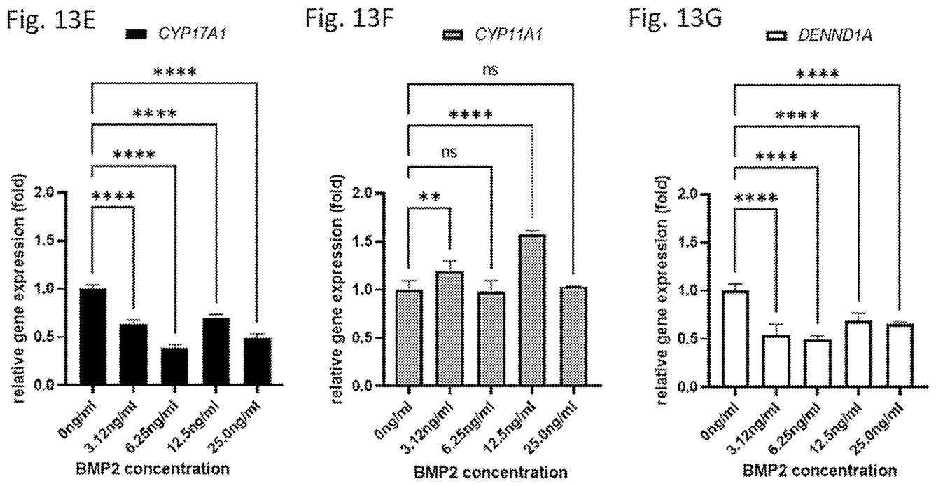

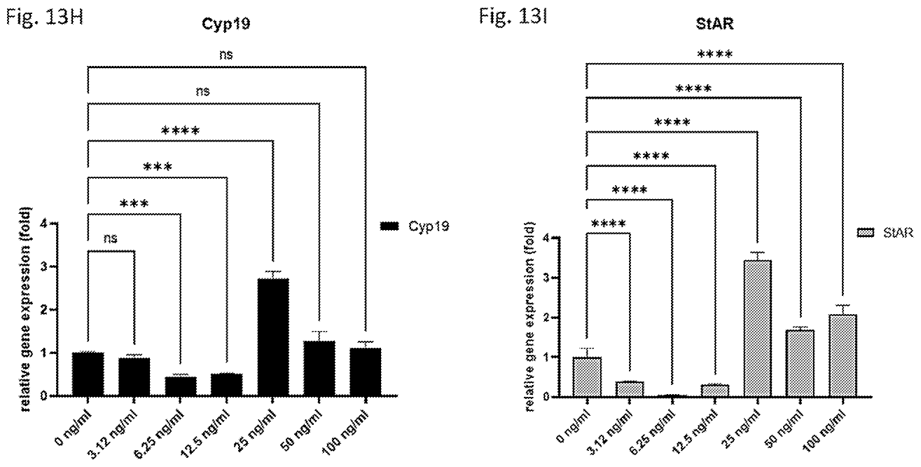

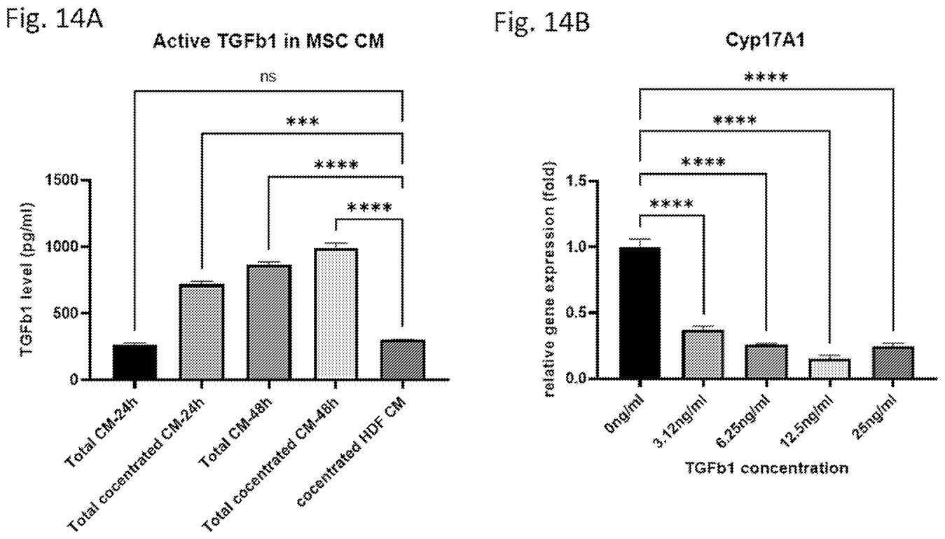

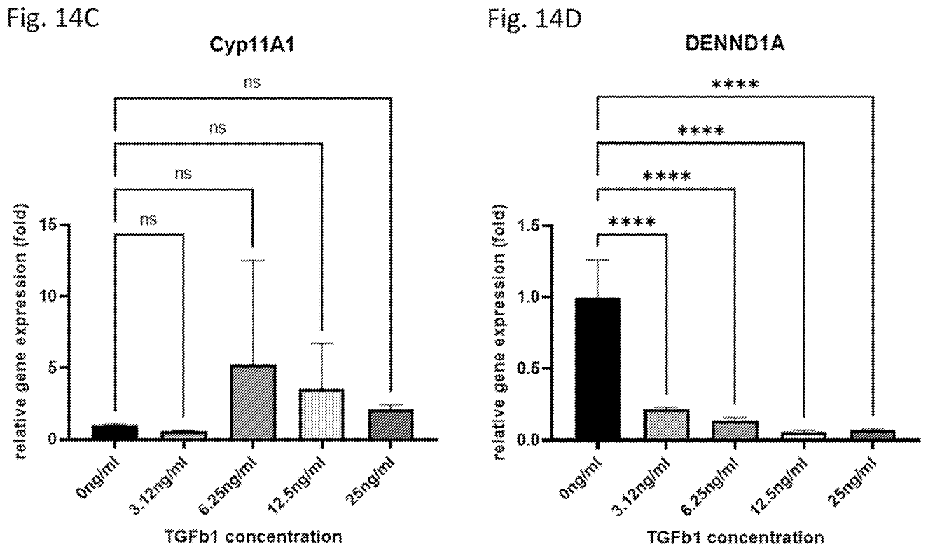

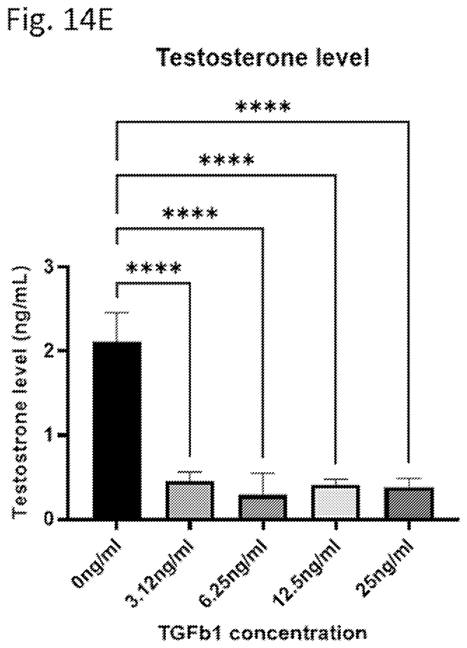

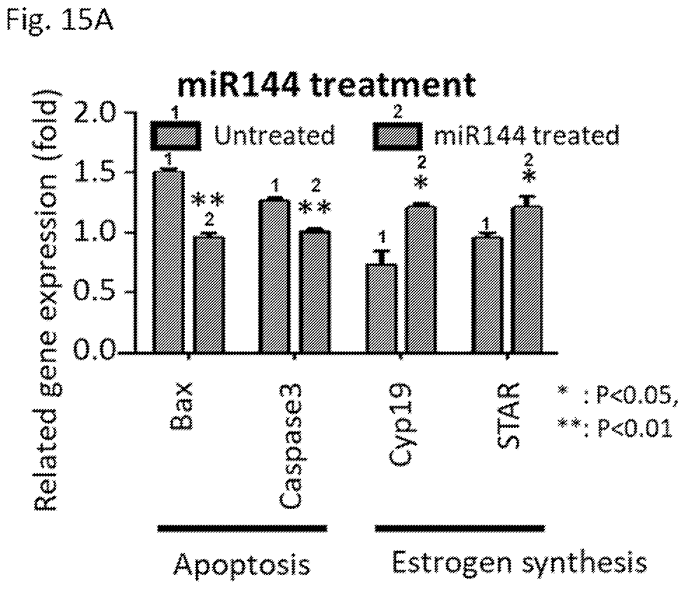

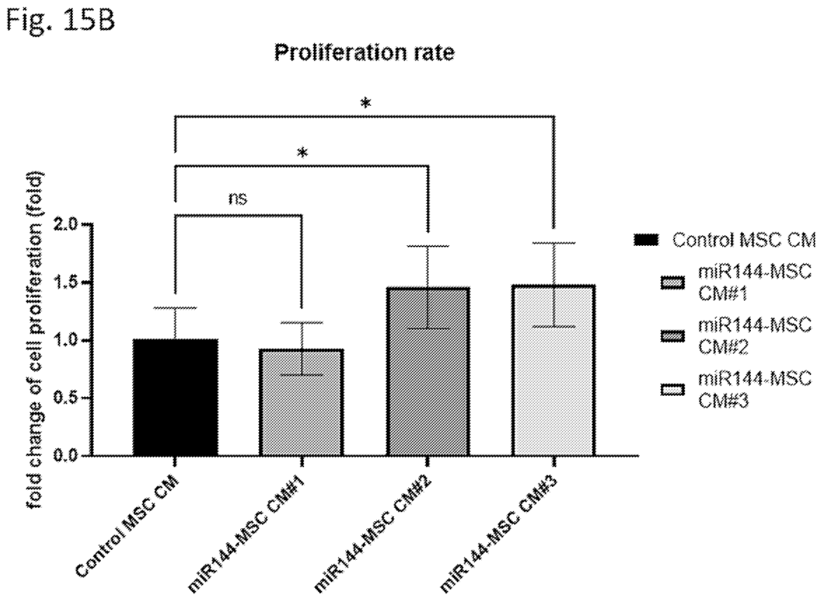

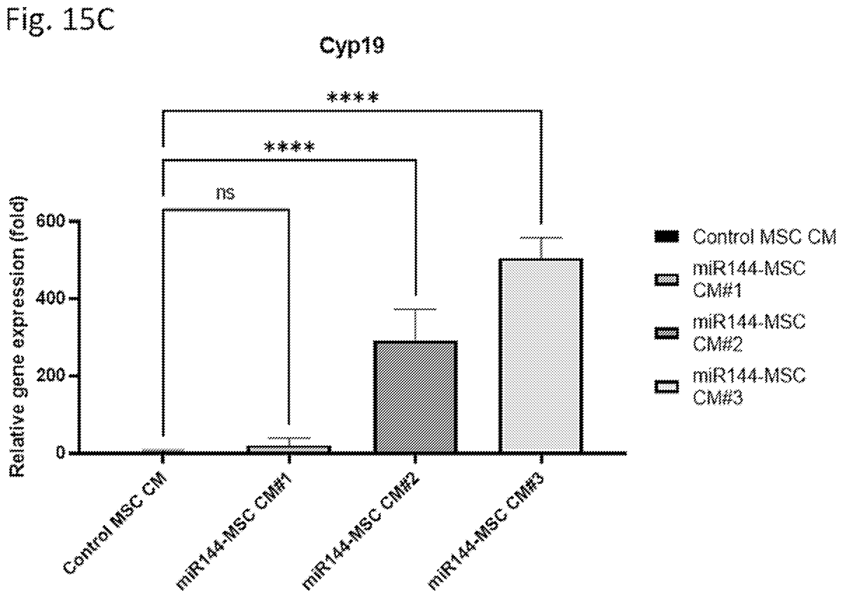

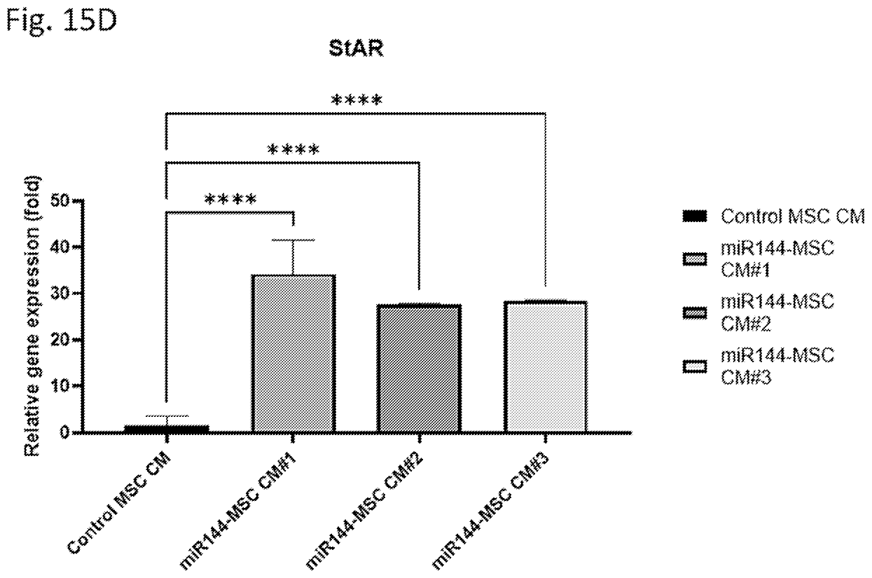

According to the data (FIG. 2, FIG. 13, FIG. 14), secretion levels of IL-10/BMP2/TGFβ1 from BM MSC are sufficient to cause some suppression of androgen production, but could be enhanced with higher concentrations of the factors. In addition, the data (FIG. 15) also suggest that overexpression of miR144 can increase the therapeutic effect of MSC secretome. In aspects, increasing expression of IL-10 by about 100% led to approximately 20-30% decrease in gene expression and proliferation rate of androgen-producing cells.

In aspects, the present disclosure provides a method of treating or preventing POI or PCOS) in a female mammal, the method comprising, consisting essentially of, or consisting of administering to the female mammal an effective amount of exosomes produced by MSCs, wherein the MSCs overexpress (a) miR144, (b) BMP-2, (c) TGFβ1, (d) IL-10, or (e) any combination of (a), (b), (c) and (d).

Exosomes are small membrane (lipid bilayer)-bound vesicles with an interior luminal space and are capable of carrying and transporting substances (e.g., DNA, protein, etc.). Exosomes may carry or transport a substance, e.g., within the lipid bilayer, at the surface of the lipid bilayer, or within the luminal space encapsulated by the lipid bilayer. Exosomes originate in endosomes, and MSC can secrete exosomes. Exosomes can have several surface markers such as CD9, CD63, CD81. Exosomes can be isolated through ultracentrifuge, ultrafiltration, or precipitation methods. Without wishing to be bound by any theory, it is believed that when an exosome contacts another membrane-bound vesicle, e.g., a cell, the membrane of the exosome fuses with the membrane of the other membrane-bound vesicle such that a substance carried/transported by the exosome can become associated with the other membrane-bound vesicle (e.g., within the fused lipid bilayer, at the surface of the fused lipid bilayer, or within the luminal space encapsulated by the fused lipid bilayer). Exosomes of MSC are commercially available and may be used in the methods as disclosed herein.

In aspects, the present disclosure provides a method of treating or preventing POI or PCOS in a female mammal, the method comprising, consisting essentially of, or consisting of administering to the female mammal an effective amount of exosomes comprising one or more effectors, wherein the one or more effectors comprises, consists essentially of, or consists of (a) miR144, (b) BMP-2, (c) TGFβ1, (d) IL-10, or (e) any combination of (a), (b), (c) and (d), and wherein the amount of the effector per exosome is greater than the amount of the effector per exosome when produced by unmodified MSCs.

In aspects, the method comprises, consists essentially of, or consists of administering to the female mammal an effective amount of any combination of MSCs or secretome from MSCs as described herein, overexpressing-exosomes produced by MSCs as described herein, and exosomes comprising one or more effectors as described herein. In aspects, exosomes can be isolated from MSC secretome and administered without what remains in the secretome. In aspects, exosomes can be isolated from MSC secretome and separately co-administered with what remains in the secretome. In aspects, exosomes can be isolated from MSC secretome and co-administered with other MSC secretome where exosomes have not been separated.

In aspects, the present disclosure provides a method of preparing mesenchymal stem cells (MSCs) for treating or preventing premature ovarian insufficiency (POI) or polycystic ovary syndrome (PCOS) in a female mammal, the method comprising, consisting essentially of, or consisting of collecting MSCs in an amount of about 4×10⁷ for administration in a single dosage.

In aspects, the present disclosure provides a method of preparing ovarian tissue-specific exosomes and/or secretome, the method comprising, consisting essentially of, or consisting of co-culturing human ovarian granulosa cells (hGrC1) with mesenchymal stem cells (MSCs).

In aspects, the present disclosure provides a method of treating or preventing premature ovarian insufficiency (POI) or polycystic ovary syndrome (PCOS) in a female mammal, the method comprising, consisting essentially of, or consisting of administering to the female mammal an effective amount of exosomes or secretome prepared according to co-culturing human ovarian granulosa cells (hGrC1) with mesenchymal stem cells (MSCs).

In aspects, the present disclosure provides a method of preventing chemotherapy-induced damage in a female mammal, the method comprising, consisting essentially of, or consisting of administering to the mammal an effective amount of exosomes and/or secretome prepared from mesenchymal stem cells (MSCs). In aspects, the damage is to a female reproductive organ. In aspects, the damage is to the endometrium. In aspects, the damage is to the ovaries.

In aspects, the present disclosure provides a method of preventing chemotherapy-induced damage in a male mammal, the method comprising, consisting essentially of, or consisting of administering to the mammal an effective amount of exosomes and/or secretome prepared from mesenchymal stem cells (MSCs). In aspects, the damage is to a male reproductive organ. In aspects, the damage is to the testes.

In aspects, the MSCs or exosomes may be isolated. In aspects, the MSCs or exosomes may be purified. By “isolated” is meant the removal of something (e.g., one or more MSCs or one or more exosomes) from its natural environment. By “purified” is meant that a given something (e.g., one or more MSCs or one or more exosomes), whether removed from nature or synthesized, has been increased in purity, wherein “purity” is a relative term, not “absolute purity.” It is to be understood, however, that MSCs or exosomes may be formulated with diluents or adjuvants and still for practical purposes be isolated. For example, MSCs or exosomes can be mixed with an acceptable carrier or diluent when used for administration.

The terms “co-administering,” “co-administration” and “co-administered” used herein refer to the administration of exosomes described herein, secretome described herein, MSCs described herein, and/or one or more additional therapeutic agents sufficiently close in time to (i) enhance the effectiveness of the exosomes, secretome, MSCs, and/or the one or more additional therapeutic agents and/or (ii) reduce an undesirable side effect of the exosomes, secretome, MSCs, and/or the one or more additional therapeutic agents. In this regard any can be administered first, and the other(s) can be administered second/sequentially. Alternatively, all can be co-administered simultaneously. In aspects, cytokine IL10 and/or cytokine BMP2 is co-administered with the exosomes.

In aspects, the MSCs, secretome, and/or exosomes can be formulated into a pharmaceutical composition. In particular, a pharmaceutical composition will comprise at least one active agent, as described herein (e.g., exosomes, secretome, and/or MSCs), and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as transdermal, subcutaneous, topical, absorption through epithelial or mucocutaneous linings, intravenous, intra-ovarian, intranasal, intra-arterial, intramuscular, intratumoral, peritumoral, interperitoneal, intrathecal, rectal, or vaginal formulations. In aspects, the pharmaceutical composition is administered intravenously. In aspects, the pharmaceutical composition is administered by intra-ovarian injection.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The active agent can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethylene glycol (e.g., PEG400), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The parenteral formulations will typically contain from about 0.5 to about 25% by weight of the active agent in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The active agent may be made into an injectable formulation. The requirements for effective pharmaceutical carriers for injectable compositions are well known to those of ordinary skill in the art. In aspects, the active agent, as exosomes, secretome, or MSCs, is suspended or resuspended (reconstituted) in normal saline, either alone or in combination. In aspects, the active agent is exosomes.

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the disclosure, the vehicle for topical application to the skin can include water, buffered solutions, alcohols, glycols such as glycerin, lipid materials such as fatty acids, mineral oils, phosphoglycerides, collagen, gelatin, and silicone-based materials.

The terms “treat,” “treating,” “treatment,” “therapeutically effective,” “prevention,” etc. used herein do not necessarily imply 100% or complete treatment/prevention/etc. Rather, there are varying degrees, which one of ordinary skill in the art recognizes as having a potential benefit or therapeutic effect. In this respect, the exosomes, secretomes, and MSCs and methods can provide any amount of any level of treatment. Furthermore, the treatment provided by the disclosed method can include the treatment of one or more conditions or symptoms of the disease or condition being treated.

In aspects, the method is a method of treating or preventing POI. In aspects, the POI is induced by exposure to chemicals. In aspects, the POI is chemotherapy-induced. In aspects, the chemotherapy-induced POI is induced by cyclophosphamide, busulfan, cisplatin or the like. In aspects, the POI is induced by chemical toxins. In aspects, the chemical toxin-induced POI is induced by dioxin. In aspects, the POI is induced by radiation exposure. In aspects, the POI is induced by radiotherapy.

In aspects, the method is a method of treating or preventing PCOS. In aspects, the method is a method of treating. In aspects, the method is a method of preventing. Treating POT or PCOS can involve restoring viability of eggs or ovaries, or can involve restoring fertility. Restoration of fertility may be to any level of fertility greater than experienced with POI or PCOS. Factors indicating greater fertility include, but are not limited to, increased ovarian follicle number, restored estrus cycle, increased pregnancy rate, increased number of offspring, and restored serum hormone aberration. In aspects, treating POI or PCOS involves restoring viability of eggs or ovaries. In aspects, treating POI or PCOS involves restoring fertility. In aspects, fertility is restored to any level of fertility greater than that experienced with POI or PCOS, wherein fertility is determined by number of follicles, pregnancy rate or delivery rate.

The disclosed methods comprise using an effective amount of exosomes, secretome, and/or MSCs. An “effective amount” means an amount sufficient to show a meaningful benefit. A meaningful benefit includes, for example, detectably treating, relieving, or lessening one or more symptoms of POI or PCOS; inhibiting, arresting development, preventing, or halting further development of POI or PCOS; reducing the severity of POI or PCOS; preventing POI or PCOS from occurring in a subject at risk thereof but yet to be diagnosed. The meaningful benefit observed can be to any suitable degree (10, 20, 30, 40, 50, 60, 70, 80, 90% or more). In aspects, one or more symptoms are prevented, reduced, halted, or eliminated subsequent to administration of exosomes or MSCs described herein, thereby effectively treating the disease to at least some degree.

One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular active agent and the desired effect. It will be appreciated by one of skill in the art that various conditions or disease states may require prolonged treatment involving multiple administrations.

The amount (e.g., therapeutically effective amount) of exosomes suitable for administration depends on, e.g., the particular route of administration and the weight of the mammal to be treated. In aspects, the number of exosomes can be from about 1E7 to about 1E13, from about 1E7 to about 1E12, from about 1E7 to about 1E11, from about 1E7 to about 1E10, from about 1E9 to about 1E13, from about 1E9 to about 1E12, from about 1E9 to about 1E11, from about 1E10 to about 1E13, from about 1E10 to about 1E12, or from about 1E11 to about 1E13. In aspects, the number of exosomes can be from about 6E10 to about 6E12. In aspects, the number of exosomes can be about 6E11. Several doses can be provided over a period of days or weeks. The amount (e.g., therapeutically effective amount) of MSCs suitable for administration depends on, e.g., the particular route of administration and the weight of the mammal to be treated. In aspects, the number of MSCs can be from about 1E3 to about 1E10, from about 1E4 to about 1E10, from about 1E4 to about 1E9, from about 1E5 to about 1E8, from about 1E7 to about 1E11, from about 1E7 to about 1E10, from about 1E5 to about 1E9, from about 1E6 to about 1E9, from about 1E6 to about 1E8, or from about 1E7 to about 1E8. In aspects, the number of MSCs can be from about 4E6 to about 4E8. In aspects, the number of MSCs can be about 4E7, e.g., for a human, and about 1E4 for a mouse. Several doses can be provided over a period of days or weeks.

The mammal may be any suitable mammal. Mammals include, but are not limited to, the order Rodentia, such as mice, and the order Lagomorpha, such as rabbits. The mammal can be from the order Carnivora, including Felines (cats) and Canines (dogs). The mammal can be from the order Artiodactyla, including Bovines (cows) and Swines (pigs) or of the order Perissodactyla, including Equines (horses). The mammal can be of the order Primates, Cebids, or Simioids (monkeys) or of the order Anthropoids (humans and apes). In aspects, the mammal is human.

The following includes certain aspects of the disclosure.

1. A method of treating or preventing premature ovarian insufficiency (POI) or polycystic ovary syndrome (PCOS) in a female mammal, the method comprising administering to the female mammal an effective amount of mesenchymal stem cells (MSCs) or secretome from MSCs, wherein the MSCs overexpress (a) miR144, (b) BMP-2, (c) TGFβ1, (d) IL-10, or (e) any combination of (a), (b), (c) and (d).

2. The method of aspect 1, wherein the method comprises administering MSCs.

3. The method of aspect 1 or 2, wherein the method comprises administering secretome from MSCs.

4. A method of treating or preventing premature ovarian insufficiency (POI) or polycystic ovary syndrome (PCOS) in a female mammal, the method comprising administering to the female mammal an effective amount of exosomes produced by mesenchymal stem cells (MSCs), wherein the MSCs overexpress (a) miR144, (b) BMP-2, (c) TGFβ1, (d) IL-10, or (e) any combination of (a), (b), (c) and (d).

5. The method of any one of aspects 1-4, wherein the MSCs overexpress miR144.

6. The method of any one of aspects 1-5, wherein the MSCs overexpress BMP-2.

7. The method of any one of aspects 1-6, wherein the MSCs overexpress TGFβ1.

8. The method of any one of aspects 1-7, wherein the MSCs overexpress IL-10.

9. A method of treating or preventing premature ovarian insufficiency (POI) or polycystic ovary syndrome (PCOS) in a female mammal, the method comprising administering to the female mammal an effective amount of exosomes comprising one or more effectors, wherein the one or more effectors comprises (a) miR144, (b) BMP-2, (c) TGFβ1, (d) IL-10, or (e) any combination of (a), (b), (c) and (d), and wherein the amount of the effector per exosome is greater than the amount of the effector per exosome when produced by unmodified mesenchymal stem cells (MSCs).

10. The method of aspect 9, wherein the one or more effectors comprises miR144.

11. The method of aspect 9 or 10, wherein the one or more effectors comprises BMP-2.

12. The method of any one of aspects 9-11, wherein the one or more effectors comprises TGFβ1.

13. The method of any one of aspects 9-12, wherein the one or more effectors comprises IL-10.

14. The method of any one of aspects 4-13, wherein the number of exosomes administered is from about 6E10 to about 6E12.

15. The method of any one of aspects 1-14, wherein the MSCs are placenta, bone marrow, or umbilical cord MSCs.

16. The method of any one of aspects 1-15, wherein cytokine IL10 is co-administered.

17. The method of any one of aspects 1-16, wherein the method is a method of treating or preventing POI.

18. The method of aspect 17, wherein the POI is chemotherapy-induced.

19. The method of aspect 18, wherein the chemotherapy-induced POI is induced by
cyclophosphamide or busulfan.

20. The method of any one of aspects 1-16, wherein the method is a method of treating or preventing PCOS.

21. The method of any one of aspects 1-20, wherein the method is a method of treating.

22. The method of any one of aspects 1-21, wherein the administration is intravenous.

23. A method of preparing mesenchymal stem cells (MSCs) for treating or preventing premature ovarian insufficiency (POI) or polycystic ovary syndrome (PCOS) in a female mammal, the method comprising collecting MSCs in an amount of about 4×10⁷ for administration in a single dosage.

24. The method of any one of aspects 1-23, wherein the female mammal is human.

25. A method of preparing ovarian tissue-specific exosomes and/or secretome, the method comprising co-culturing human ovarian granulosa cells (hGrC1) with mesenchymal stem cells (MSCs).

26. A method of treating or preventing premature ovarian insufficiency (POI) or polycystic ovary syndrome (PCOS) in a female mammal, the method comprising administering to the female mammal an effective amount of exosomes or secretome prepared according to aspect 25.

27. The method of aspect 26, wherein the female mammal is human.

28. A method of preventing chemotherapy-induced damage in a male mammal, the method comprising administering to the mammal an effective amount of exosomes and/or secretome prepared from mesenchymal stem cells (MSCs).

29. The method of aspect 28, wherein the male mammal is a human.

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following examples further illustrate aspects of the disclosure, but, of course, should not be construed as in any way limiting its scope.

### Example 1

### Materials and Methods

### Human Bone-Marrow Mesenchymal Stem Cell Culture

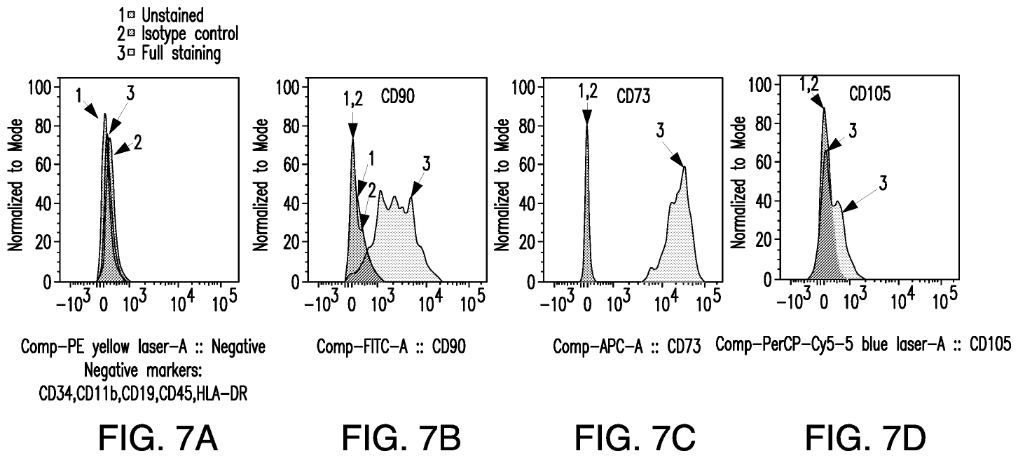

Human BM-hMSC (Passage 2) were purchased from Lonza, USA (PT #2501). These cells were isolated from the bone marrow of a healthy non-diabetic female donor 32-year-old. The cells were cultured in mesenchymal stem cell growth medium (MSCGM) per the manufacturer's recommended expansion protocol. When the culture reached approximately 80% confluence, cells were trypsinized using a 0.05% trypsin-EDTA solution and serially expanded for use in experiments. Cells were characterized for typical BM-MSC-positive (CD90, CD73, CD105) and negative (CD34, CD11b, CD19, CD45, HLA-DR) cell surface markers using the human MSC analysis kit (BD Stemflow®, CA, USA cat. no. 562245). See FIGS. 7A-7D.

### Human Adrenocortical-Carcinoma Cell Line Culture

Human adrenocortical-carcinoma cells (H295R cells) were used as an in vitro cell culture model for androgen production. H295R cells were purchased from ATCC (Manassas, VA, USA, cat. no. ATCC® CRL-2128™) and cultured per the recommended guidelines. Briefly, H295R cells were cultured in flasks pre-coated with extracellular matrix (Gibco, USA, cat. no. S-006-100) with DMEM/F12 (Gibco, cat. no. 21041025) and 2.5% Nu-Serum (Corning, USA). The cells were subcultured at a ratio of 1:3 to 1:4 and culture media were changed twice a week.

Human Theca Cell Culture from Women with PCOS

Human theca interna tissue was collected at the time of oophorectomy (n=2), which was performed as clinically indicated using a protocol (Nelson et al., Endocrinol. Metab., 86: 5925-5933 (2001); McAllister, Endocrine, 3: 143-149 (1995); McAllister et al., Proc. Natl. Acad. Sci. USA, 111: E1519-1527 (2014); and Nelson et al., Mol. Endocrinol., 13: 946-957 (1999); each of which is incorporated by reference herein) approved by an Institutional Review Board. Theca cells from PCOS ovarian follicles were isolated and cultured as previously reported (Nelson-Degrave et al., Mol. Endocrinol., 19: 379-390 (20050 and Wickenheisser et al., PLoS One, 7: e48963 (2012); each of which is incorporated by reference herein). The follicles were isolated from the ovaries and dissected under a microscope in a dish containing a 1:1 mixture of DMEM and Ham's F12 medium supplemented with 10% fetal bovine serum (FBS). The cleaned theca shells were digested with 0.05% collagenase I, 0.05% collagenase IA, and 0.01% deoxyribonucleic, in medium containing 10% FBS. The isolated cells were cultured in dishes pre-coated with fibronectin in a 1:1 mixture of DMEM and Ham's F12 medium containing 10% FBS, 10% horse serum, 2% UltroSer G, 20 nm insulin, 20 nm selenium, 1 μM vitamin E, and antibiotics. Experiments were performed using passage 4 (31-38 population doublings) PCOS theca cells.

### Preparation of the BM-hMSC Secretome

The secretome was prepared from three to five passages of BM-hMSC in T75 flasks. Media were collected and discarded from the BM-hMSC culture at 80-90% confluence. Cells were then washed three times with phosphate-buffered saline (PBS) for complete removal of serum. Cells were then maintained in DMEM/F12 (Gibco, USA) serum-free media for 24 hours to collect the secretome. After 24 hours, the media were collected, centrifuged at 500 g for 5 min at 4° C. to remove the cell debris, aliquoted, and stored at −80° C. for use in experiments. DMEM/F12 serum-free media without cells were incubated for 24 hours in the T75 cell culture flask to serve as a negative control.

For in vivo experiments, the secretome was collected using the above method, and cultured cells were trypsinized from the flasks and counted. The average cell count was 2.25×10⁶ cells per flask. The collected BM-hMSC media were then aliquoted at a volume calculated based on the cell secretions from 5×10⁵ cells on average per ovary of each mouse. The media/secretome were concentrated using a vacuum concentrator (Labconco, MO, USA) and stored at −80° C. for use in in vivo experiments. Before intra-ovarian injection, the concentrated secretome was reconstituted with PBS to a final volume of 10 μl per ovary.

Treatment of H295R Cells and Human PCOS Theca Cells with the BM-hMSC Secretome

H295R cells and human PCOS theca cells were cultured separately on pre-coated six-well plates for 48 hours. Cells were then treated for 24 hours with secretome diluted in basal media (serum-free) at a 1:1 ratio. Cell culture media were replaced with serum-free media or secretome media, and cells were incubated for an additional 24 hours. After the incubation period, cells were collected for analysis of steroidogenesis-related gene expression. Cell culture media was used for hormone quantification using an automated chemiluminescence immunoassay system, UniCel DxI 800, Access Immunoassay System (Beckman Coulter Inc., CA, USA) (Hernandez et al., Endocrinol. Nutr., 58: 50-51 (2011), incorporated by reference herein).

Treatment of H295R Cells with Recombinant Human IL-10

To investigate the anti-inflammatory effect of the BM-hMSC secretome, the amount of IL-10 secreted by BM-hMSC into the culture media was measured by ELISA (Abcam, Cambridge, MA, USA) following the manufacturer's instructions (Choi et al., Clin. Exp. Immunol., 153: 269-276 (2008), incorporated by reference herein). The effect of IL-10 on steroidogenesis-related gene expression, androgen secretion, and pro-inflammatory marker expression in H295R cells was explored after treatment with 0, 125, 250, or 500 μg/ml recombinant human IL-10 (rhIL-10; R & D Biosystem, Cat No. 217-IL-010). These concentrations were selected based on the previously reported level of IL-10 secreted by hMSCs (Qu et al., Exp. Hematol., 40: 761-770 (2012), incorporated by reference herein). H295R cells were then collected for gene expression analysis, and cell culture media were used for measurement of testosterone using an automated chemiluminescence immunoassay system, UniCel DxI 800, Access Immunoassay System (Beckman Coulter Inc., CA, USA) (Hernandez et al., Endocrinol. Nutr., 58: 50-51 (2011), incorporated by reference herein) and androstenedione using ELISA (Biovision, CA, USA) (Fox et al., Endocrinology, 160: 2946-2958 (2019), incorporated by reference herein).

### PCOS Mouse Model and Intra-Ovarian Injection of BM-hMSC

Three-week-old female C57BL/6 mice (Charles River, MA, USA) were housed in a vivarium for 1 week under specific pathogen-free conditions. The animal experiment protocol for this study was approved, and all animal experiments were performed in compliance with policies and guidelines for use of laboratory animals.

At 4 weeks of age, mice (n=10/group) were subcutaneously implanted with a placebo or 5 mg LTZ pellet (Innovative Research of America, Sarasota, FL, USA), which provides a constant release of LTZ (50 pg/day). Body weight was monitored weekly before and post-implantation. Body weight and insulin resistance (measured by glucose tolerance test, GTT) were used to monitor development of PCOS characteristics.

Five weeks after placebo or LTZ pellet implantation, mice underwent intra-ovarian injection of BM-hMSC via laparotomy. Mice were treated preoperatively with a single dose of buprenorphine (0.1 mg/kg) and were kept under anesthesia with 1-4% inhalation of isoflurane during the entire procedure. A single midline incision, less than 25 mm, was made on the skin to access both ovaries via the caudal abdominal cavity. For the BM-hMSC group, cells were injected in both ovaries at a concentration of 5.0×10⁵ cells per ovary resuspended in 10 μl PBS. For the secretome group, concentrated secretome reconstituted in 10 μL PBS was injected per ovary in both ovaries. For the control group, 10 μl of PBS was injected into both ovaries. The incision was closed by suturing, followed by wiping with a clean disinfectant swab. Two weeks after BM-hMSC engraftment or secretome injection, the mice were anesthetized and gonadal fat pads, brown fat, and ovaries were collected. A portion of the gonadal and brown fat, as well as one ovary, were fixed in 4% paraformaldehyde and embedded in paraffin; the remainder of the tissue and the other ovary was frozen at −80° C. for further analysis.

### Glucose Tolerance Test

Glucose tolerance testing was performed on mice 5 weeks after placebo or LTZ pellet implantation and 2 weeks after BM-hMSC engraftment or secretome treatment. Mice were fasted for 16 h (5 p.m. to 9 a.m.), with free access to drinking water, after which they received an intra-peritoneal (i.p.) injection of D-glucose (2.0 g/kg body weight). Blood glucose level was measured at 0, 15, 30, 60, 90, and 120 min following glucose injection using a Bayer glucose monitor (Roche Diagnostics Corp, IN, USA).

### Indirect Calorimetry

Metabolic rate was measured in mice at 11 weeks of age by indirect calorimetry in open-circuit Oxymax chambers, a unit of the Comprehensive Lab Animal Monitoring System (CLAMS; Columbus Instruments, state, USA). Two weeks after BM-hMSC treatment, mice receiving LTZ only or LTZ and treated with BM-hMSC (n=3) were acclimated to calorimetry cages for 2 days before data sampling at 23° C. under 12:12 hours light:dark cycle. Oxygen consumption rate (VO₂), carbon dioxide release (VCO₂), respiratory exchange ratio (RER), and heat production were measured in individual mice. The horizontal activity was measured on x, y, and z-axes.

### Serum Hormone Measurements

Blood was collected from all the groups by cardiac exsanguination under isoflurane anesthesia; serum was separated and stored at −80° C. Serum hormone levels were measured. Serum testosterone (T) and estradiol (E2) levels were measured using ELISA. Serum luteinizing hormone (LH) and follicle-stimulating hormone (FSH) levels were measured by radioimmunoassay (RIA). The sensitivities of each assay are 10 ng/dL (T), 3 μg/ml (E2), 3 ng/ml (FSH), and 0.04 ng/ml (LH). Serum cytokines were analyzed in a membrane-based antibody array (Ray Biotech, GA, USA) per the manufacturer's protocol.

### Breeding Experiments

One week after BM-hMSC engraftment or secretome treatment, 6 mice per group were randomly selected for the breeding experiment. One male C57BL/6 breeder mouse was used for every two female mice. The male and female mice were caged together for 10 days. Mating was determined by the presence of sperm plug in the vagina. Most of the female mice showed a sperm plug within 3 days, and the average number of pups from each female mouse was compared between treatment groups. At the end of the experiment, all delivered pups were counted per group, their body weight was measured, and any morphological anomalies were noted.

### Histology and Immunohistochemistry

Ovaries and fat tissues were collected, fixed in 4% paraformaldehyde, and embedded in paraffin blocks. Tissue sections were stained with hematoxylin-eosin (H&E) and murine anti-UCP-1 (Abcam, MA, USA), followed by detection with a biotin-labeled rabbit anti-rat antibody and staining with the ABC kit (Vector Laboratories, Burlingame, CA, USA). Sample processing and staining were performed. Histological analyses were performed using Asperio ImageScope (Leica Biosystem, Wetzlar, Germany).

### Immunoblot Analysis

Following treatment of H295R cells and human PCOS theca cells with the BM-hMSC secretome, and treatment of mice with BM-hMSC or its secretome, cultured cells and collected ovarian tissue were lysed with RIPA buffer (Cell Signaling, MA, USA) containing protease and phosphatase inhibitor cocktail (Thermo Fisher Scientific Inc., MA, USA) and sonicated at 20 amplitude with 5 sec on and 5 sec off for a 1-minute cycle. Sonicated samples were then centrifuged at 12,000 rpm for 5 min and the supernatant was transferred into separate tubes. The protein concentration of all samples was determined by the Bradford method. For immunoblot analysis, samples containing equal amounts of protein were incubated with 1× gel loading buffer and separated by SDS-PAGE (4-20% criterion, Bio-Rad), then transferred to PVDF membrane using a Trans-blot turbo system (Bio-Rad, Hercules, CA, USA). After protein transfer, blocked membranes were incubated in 1% non-fat dry milk in 1×PBS (0.05% Tween) overnight at 4° C., with primary antibodies against CYP17A1 (ab125022, 1:500 dilution, Abcam), CYP11A1 (ab75497, 1:500 dilution, Abcam), DENND1A (LS-C167356, 1:250, LSBio), VEGFA (ab1316, 1:500 dilution, Abcam), or (3-actin (clone AC-15, A5441, 1:5000, Sigma) in 1% non-fat dry milk in 1×PBS with 0.05% Tween overnight at 4° C. After washing, the membrane was incubated with the appropriate HRP-linked secondary antibodies (anti-mouse secondary antibody, cat. no. 7076, 1:5000 or anti-rabbit secondary antibody, cat. no. 7074, 1:3000, Cell Signaling) in 5% non-fat dry milk in 1×PBS with 0.1% Tween at room temperature for 1 hour. The membrane was developed with Trident Femto Western HRP substrate (GeneTex, Irvine, CA, USA) and visualized using the ChemiDoc XRS+molecular imager (Bio-Rad, Hercules, CA, USA). After imaging, membranes were stripped with Restore™ PLUS stripping buffer (Thermo Scientific, MA, USA) to incubate with another antibody. The signal density of each protein band was quantified using Image J software (US National Institute of Health, Bethesda, MD, USA) and normalized against the corresponding R-actin band.

Quantitative Real-Time PCR (qRT-PCR)

RNA was extracted from H295R cells and human PCOS theca cells treated with the BM-hMSC secretome or rhlL-10. RNA was also extracted from fat and ovarian tissues collected from mice treated with BM-hMSCs or the BM-hMSC secretome. RNA extraction was done using TRIzol (Invitrogen, USA) according to the manufacturer's instructions. The concentration and purity of the extracted RNA were checked using a NanoDrop spectrometer (Thermo Scientific, MA, USA). 1 μg of total RNA was reverse transcribed using RNA to cDNA EcoDry™ Premix (Double Primed) (Takara Bio USA Inc., CA, USA). The reaction mixture was incubated for 1 h at 42° C.; incubation was stopped at 70° C. for 10 min. Quantitative real-time PCR (qPCR) was performed using the CFX96 PCR instrument and SYBR Green Supermix (Bio-Rad, Hercules, CA, USA) with specific primers to the target genes in a 20 μL final reaction volume. The primer sequences are listed in Table 1. Beta-actin was used as a reference gene for sample normalization. The delta-delta threshold cycle (ΔΔCt) method was used to calculate the fold change expression in mRNA level in the samples.

TABLE 1

| | | Seq | | Seq |
|---|---|---|---|---|
| | | ID | | ID |
| Gene | Forward primer (5′-3′) | No: | Reverse primer (5′-3′) | No: |
| Human ACTB | TGGATCAGCAAGCAGGAGTATG | 2 | GCATTTGCGGTGGACGAT | 3 |
| Human CYP17A1 | GGCCTCAAATGGCAACTCTAGA | 4 | CTTCTGATCGCCATCCTTGAA | 5 |
| Human CYP11A1 | GAGGGAGACGGGCACACA | 6 | TGACATAAACCGACTCCACGTT | 7 |
| Human DENND1A | CAATTCCCGGAGGACTACAGT | 8 | AGCACGAATGTGAAGTTCTGG | 9 |
| Human IL6 | TGCACTTTATGACGCACTCAC | 10 | TGTCCAAAAACACGAAATCATGC | 11 |
| Human IL1B | ATGATGGCTTATTACAGTGGCAA | 12 | GTCGGAGATTCGTAGCTGGA | 13 |
| Human TNFA | AAGCACACTGGTTTCCACACT | 14 | TGGGTCCCTGCATATCCGTT | 15 |
| Mouse Gapdh | CACATTGGGGGTAGGAACAC | 16 | AACTTTGGCATTGTGGAAGG | 17 |
| Mouse Cyp17a1 | GAGTTTGCCATCCCGAAGGA | 18 | CCAGCTCCGAAGGGCAAATA | 19 |
| Mouse Cyp19a1 | TTTCGCTGAGAGACGTGGAG | 20 | AGGATTGCTGCTTCGACCTC | 21 |
| Mouse Fshr | GTGCATTCAACGGAACCCAG | 22 | TCTAAGCCATGGTTGGGCAG | 23 |
| Mouse Vegfa | GTACCTCCACCATGCCCAGT | 24 | GCATTCACATCTGCTGTGCT | 25 |
| Mouse Il1b | TTCAGGCAGGCAGTATCACTC | 26 | GAAGGTCCACGGGAAAGACAC | 27 |
| Mouse Il6 | ATCCAGTTGCCTTCTTGGGACTGA | 28 | TAAGCCTCCGACTTGTGAAGTGGT | 29 |
| Mouse Il10 | CGGGAAGACAATAACTGCACCC | 30 | CGGTTAGCAGTATGTTGTCCAGC | 31 |
| Mouse Ccl2 | AGATGCAGTTAACGCCCCAC | 32 | ACCCATTCCTTCTTGGGGTC | 33 |
| Mouse Cd11c | ACGTCAGTACAAGGAGATGTTGGA | 34 | ATCCTATTGCAGAATGCTTCTTTACC | 35 |
| Mouse Tnfa | TCCCAGGTTCTCTTCAAGGG | 36 | GGTGAGGAGCACGTAGTCGG | 37 |
| Mouse Ki67 | CTGCCTGCGAAGAGAGCATC | 38 | AGCTCCACTTCGCCTTTTGG | 39 |
| Mouse Erb | TTCCCGGCAGCACCAGTAACC | 40 | TCCCTCTTTGCGTTTGGACTA | 41 |
| Mouse Ar | TTGCAAGAGAGCTGCATCAGTT | 42 | ACTGTGTGTGGAAATAGATGGGC | 43 |
| Mouse Aib1 | GCCTGGCTTTGAAGACATAATCCG | 44 | TCTTGATAGTGACGCTTCTGGGAC | 45 |
| Mouse | GGCCCATCATGCTATGGAAC | 46 | GTGAGGGATCACTCGCCATC | 47 |
| Adiponectin | | | | |
| Mouse Leptin | TTGCCATATTCCTCACCA | 48 | CCCACTCGATCAGATGTGCTC | 49 |
| Mouse Ucp1 | CAAAAACAGAAGGATTGCCGAAA | 50 | TCTTGGACTGAGTCGTAGAGG | 51 |
| Mouse Pgc1a | TATGGAGTGACATAGAGTGTG | 52 | GTCGCTACACCACTTCAATCC | 53 |
| Mouse Cidea | TGACATTCATGGGATTGCAGAC | 54 | CATGGTTTGAAACTCGAAAAGGG | 55 |
| Mouse Prdm16 | CCACCAGCGAGGACTTCAC | 56 | GGAGGACTCTCGTAGCTCGAA | 57 |

### Flow Cytometry (FACS) Analysis

After treatment with the BM-hMSC secretome or basal media control, H295R cells were analyzed by FACS for proliferation, apoptosis, and inflammatory markers using antibodies against Ki67 antibody (BioLegend, Cat no. 350514), Annexin-V (BioLegend, Cat no. 640919), IL-10 (R&D Systems, Cat no. IC8406A), and TNF-α (BioLegend, Cat no. 502943). In brief, treated cell pellets were harvested and fixed/permeabilized with BD cytofix/cytoperm kit reagent (BD Bioscience, CA, USA) for intracellular staining, per the manufacturer's instructions. After centrifugation at 1500 rpm for 5 minutes, a total of 1×10⁶ cells were resuspended in 200 μl of antibody solution and incubated for 30 min at room temperature in the dark. After washing, the cells were resuspended in PBS with 2% FBS (v/v) for FACS analysis using (BD, Gallios, Flow-cytometer). Data were analyzed using FlowJo software.

### Statistical Analysis

Comparisons between groups were made by one-way ANOVA with Tukey's post hoc test or Student's t-tests. All data are presented as mean±standard deviation (SD). A difference between groups with *p<0.05, **p<0.005, or ***p<0.0005 was considered statistically significant.

Treatment of H295R Cells with BMP-2

H295R cells were cultured on six-well plates precoated with an extracellular matrix at a density of 18×10⁴ cells per well and cultured for 48 h. Cells were treated with 0 to 50 ng/mL recombinant human BMP-2 (R & D Systems, Minneapolis, MN, USA) in H295R culture media for 48 h. After removal of treatment media, the cells were washed with PBS three times before adding basal media (serum-free) and incubating for another 24 h. To compare the cell number, cells were determined using a CTS™ TrypLE select enzyme (Gibco, a division of Thermo Fisher Scientific, Waltham, MA, USA). The number of live cells was counted by Trypan blue assay, which is the most widely used and still the gold standard method to perform cell viability assays in cell culture. Cells were collected to analyze the expression of steroido-genesis pathway genes. The cell culture supernatant was used for chemiluminescent quantification of testosterone released by H295R cells using an automated UniCel DxI 800 Access Immunoassay System (Beckman Coulter, Inc., Brea, CA, USA).

### Results

### BM-hMSC Secretome Elicits Anti-Proliferative and Apoptotic Effects in H295R Cells

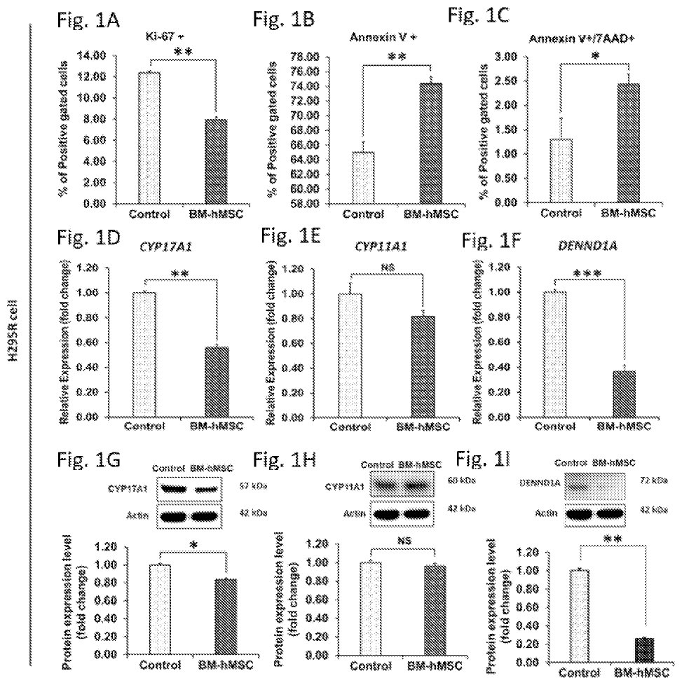

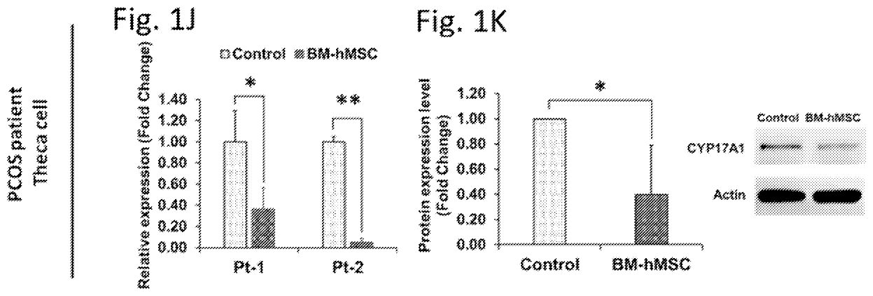

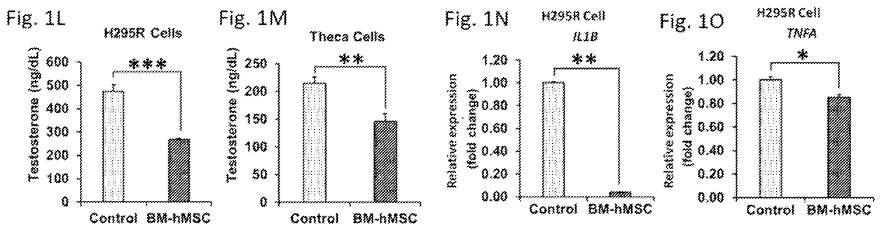

H295R cells were incubated with BM-hMSC secretome to evaluate therapeutic potential. After 24 hours, a significant reduction (7.96%±0.23) was observed in cell growth rate, as measured by Ki-67 protein expression compared with control media-treated cells (12.37%±0.19; FIG. 1A). Additionally, secretome treatment significantly increased both early apoptosis (74.38%±1.00; FIG. 1B) as well as late apoptosis and necrosis (2.43%±0.21; FIG. 1C), as measured by Annexin-V and Annexin-V/7-AAD expression, respectively, compared with the control group (64.94%±1.47 and 1.30%±0.43). Thus, the results indicate that BM-hMSC secretome inhibits growth of H295R cells.

### BM-hMSC Secretome Decreases Steroidogenesis-Related Gene Expression and Androgen Production in H295R Cells

CYP17A1, CYP11A1, and DENND1A, key genes for ovarian androgen biosynthesis, are upregulated in PCOS-theca cells compared with healthy theca cells. The effect of the BM-hMSC secretome on the expression of these genes was evaluated using the in vitro model. Secretome treatment resulted in significant downregulation of CYP17A1 (0.56±0.02 fold) and DENND1A (0.37±0.05 fold) gene expression in H295R cells compared with media-treated cells (FIGS. 1D-1F). However there was no significant decrease in CYP11A1 gene expression (0.82±0.05 fold, p=0.127). These findings were confirmed at the protein level using immunoblot analysis, which showed that CYP17A1 (0.84±0.02 fold) and DENND1A (0.26±0.01 fold) were significantly decreased in secretome-treated H295R cells compared with the control group, while no change was observed in CYP11A1 (0.97±0.02 fold, p=0.29; FIGS. 1G-1I). These observations were validated in PCOS patient-derived theca cells (n=2) treated with BM-hMSC secretome. Secretome treatment significantly downregulated CYP17A1 (Patient 1: 0.36±0.20 fold, Patient 2: 0.05±0.04 fold) gene expression (FIG. 1J) and protein expression (0.40±0.39 fold; FIG. 1K) in theca cells from both patients compared with media-treated controls.

The effects of steroidogenesis-related gene inhibition by the BM-hMSC secretome on testosterone secretion was investigated. Whether steroidogenesis-related gene inhibition by the BM-hMSC secretome affected testosterone secretion was explored. Compared with the media control group (474.6±27.5 ng/dL), secretome treatment suppressed testosterone secretion in H295R cells (267.7±4.0 ng/dL) (FIG. 1L). Testosterone secretion was also suppressed in human PCOS theca cells (146.4±13.4 ng/dL) (FIG. 1M) compared with the media control group (214.7±11.8 ng/dL). In summary, the data indicate that BM-hMSC secreted factors inhibit androgen production.

### BM-hMSC Secretome Exerts an Anti-Inflammatory Effect on H295R Cells

Chronic inflammation is a major factor affecting the ovarian microenvironment in patients with PCOS inducing higher ovarian androgen production, that involves two pro-inflammatory cytokines, interleukin-1 beta (IL-1β) and tumor necrosis factor (TNF-α). Treatment of H295R cells with BM-hMSC secretome significantly downregulated gene expression of IL-1β (IL1B: 0.04±0.003 fold) and TNF-α (TNFA: 0.85±0.02 fold) compared with the control media group (FIGS. 1N, 1O), indicating a decreased inflammatory response after treatment.

### IL-10 Decreases Steroidogenesis-Related Gene Expression and Androgen Production in H295R Cells

Based on the observed anti-inflammatory effect of the BM-hMSC secretome, the effect of the anti-inflammatory cytokine, IL-10, which is known to be released by MSC was tested. IL-10 exerts immune-suppressive and anti-inflammatory effects in several disorders, including PCOS. There is a significantly lower serum level of IL-10 in women with PCOS compared with age- and BMI-matched healthy controls. High IL-10 levels may also increase insulin sensitivity by ameliorating the inflammatory responses to TNF-α and IL-6, which contribute to insulin resistance in PCOS. First, IL-10 secretion from BM-MSCs was explored by ELISA of conditioned media. A high concentration of IL-10 (164.2±1.42 pg/ml) was found compared with control media (1.32±0.15 pg/ml; FIG. 2A). Next examined was the effect of IL-10 on steroidogenesis-related gene expression and androgen production in H295R cells. As shown in FIG. 2, recombinant human IL-10 treatment significantly downregulated the expression of CYP17A1 including at the lowest dose of 125 pg/ml of concentration (0.91±0.01 fold) (FIG. 2B). The same IL-10 treatment condition (125 pg/ml) also significantly decreased CYP11A1 gene expression (0.84±0.02 fold) and DENND1A gene expression (0.87±0.01 fold) in H295R cells (FIG. 2C, D). Additionally, testosterone level in conditioned media from IL-10 treated H295R cells was analyzed. Testosterone level was not significantly decreased in 125 pg/ml of IL-10-treated H295R cells (1.52±0.01 ng/ml, p=0.789) compared to control (1.56±0.02 ng/ml). However, treatment at higher concentration of IL-10 (500 pg/ml) significantly decreased testosterone level in H295R cells (1.32±0.10 ng/ml, p=0.02) (FIG. 2E). The androstenedione level (Control: 1.55±0.03 ng/ml) also did not decrease significantly at 125 pg/ml of IL-10 treated H295R cells (1.44±0.04 ng/ml, p=0.15), but significantly decreased at higher concentrations (250 pg/ml: 1.34±0.04 ng/ml, 500 pg/ml: 1.38±0.10 ng/ml) (FIG. 2F). The data suggest that IL-10 inhibits androgen production by regulating steroidogenic gene expression in a dose-dependent manner.

### IL-10 Exerts an Anti-Inflammatory Effect on H295R Cells

Next, the anti-inflammatory effects of IL-10 on H295R cells was explored by measuring expression of key pro-inflammatory cytokines, IL-6, TNF-α, and IL-1β, following IL-10 treatment. All tested concentrations of IL-10 significantly downregulated IL6 (Control: 1.00±0.08 fold, 125 μg/ml: 0.73±0.03 fold, 250 μg/ml: 0.79±0.02 fold, 500 μg/ml: 0.70±0.11 fold), TNFA (Control: 1.00±0.04 fold, 125 μg/ml: 0.44±0.02 fold, 250 μg/ml: 0.59±0.07 fold, 500 μg/ml: 0.54±0.04 fold), and IL1B (Control: 1.00±0.04 fold, 125 μg/ml: 0.84±0.02 fold, 250 μg/ml: 0.60±0.08 fold, 500 μg/ml: 0.76±0.1 fold) gene expression compared with untreated controls (FIGS. 2G-2I). Together, these data suggest that IL-10 is a key mediator of the effect of the BM-hMSC secretome on in vitro human cell PCOS models.

### BM-hMSC Reverse the Metabolic Phenotypes in an LTZ-Induced PCOS Mouse Model

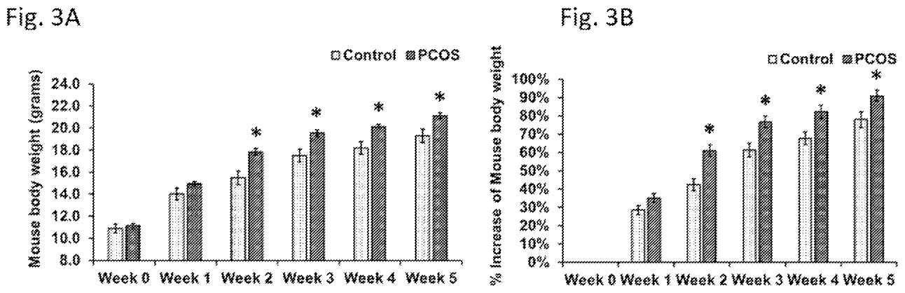

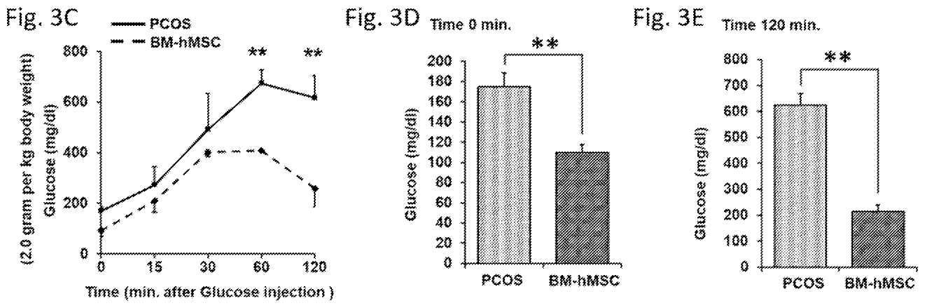

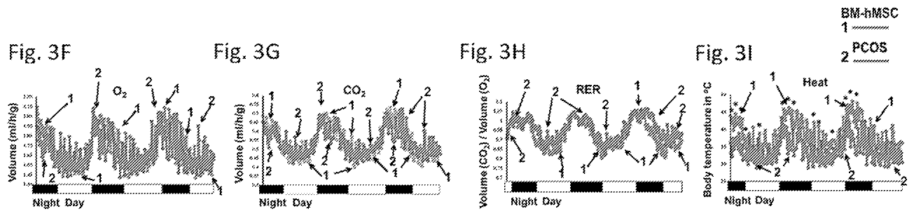

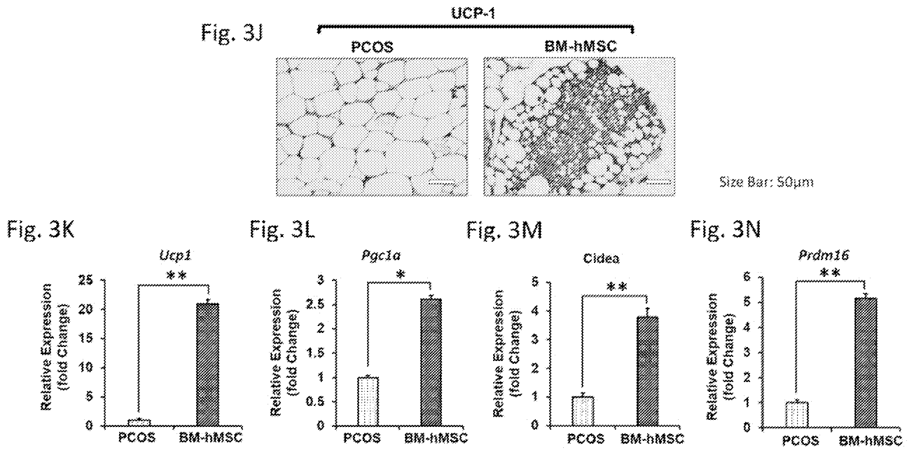

Next, the potential therapeutic effects of BM-hMSC in vivo was evaluated by injecting BM-hMSC into the ovaries of the LTZ-induced PCOS mouse model (Kauffman et al., Biol. Reprod., 93: 69 (2015), incorporated herein by reference). Five weeks after LTZ implantation, the mice in the PCOS group were significantly heavier (21.1±0.25 grams) compared with age-matched control mice (19.3±0.60 grams) that had received placebo pellets (FIGS. 3A, 3B). Since PCOS women have insulin resistance and impaired glucose tolerance, also performed was a glucose tolerance test (GTT) and measured energy expenditure in PCOS mice before (5 weeks after LTZ or placebo) and 2 weeks after BM-hMSC engraftment (7 weeks after LTZ or placebo). PCOS mice treated with BM-hMSC exhibited a normal glucose tolerance profile compared with untreated PCOS mice (FIGS. 3C-3E). Moreover, it was found that the untreated PCOS group had lower energy expenditure, based on a significant difference in thermogenesis, compared with PCOS mice treated with BM-hMSC (FIGS. 3F-3I).

The increase in thermogenesis in BM-hMSC-treated PCOS mice encouraged further evaluation of fat metabolism in treated versus control PCOS mice. Brown fat cells play a role in the regulation of total energy expenditure. A process called “browning,” which refers to the transition of white fat into brown fat, is associated with upregulation of UCP-1. Therefore, white fat tissues collected from BM-hMSC-treated and untreated PCOS mice were stained with UCP-1 and greater proportions of brown-like fat cells were found, suggesting increased browning of white fat, in the BM-hMSC-treated group (FIG. 3J). At the molecular level, the browning process is regulated by several genes that control multiple aspects of mitochondrial activity, such as Pgc-1α, Cidea, and Prdm-16. qPCR confirmed the UCP-1 immunohistochemistry results and showed significant upregulation of Ucp1 (21.00±0.67 fold), Pgc1a (2.61±0.08 fold), Cidea (3.78±0.31 fold), and Prdm16 (5.15±0.19 fold) gene expression in white fat collected from the BM-hMSC-treated PCOS mice compared with the untreated PCOS mice (FIGS. 3K-3N).

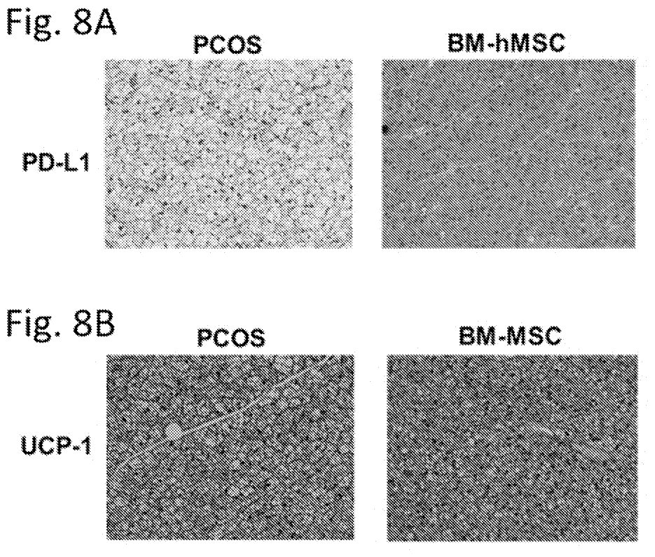

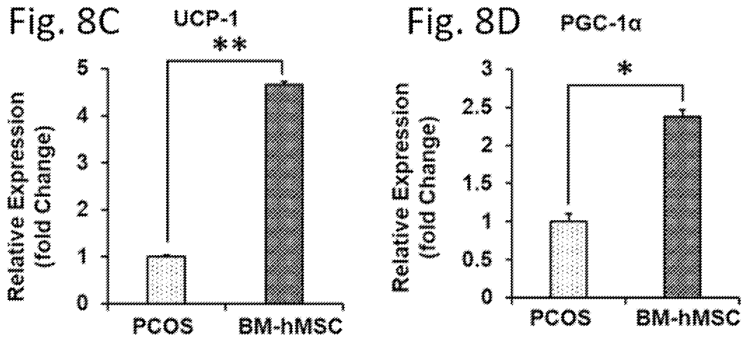

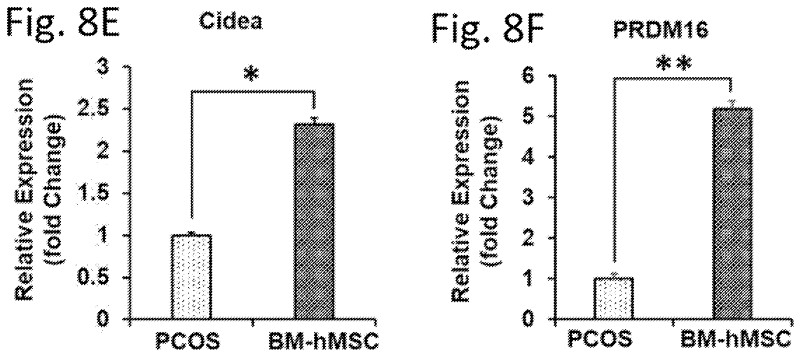

Marker expression levels in brown fat tissue were explored, and found that BM-hMSC treatment increases brown fat-related marker expression even in brown fat tissue (FIGS. 8A-8F). These results suggest that BM-hMSC can regulate adipose tissue metabolism by ameliorating inflammation and promoting brown fat formation.

### BM-hMSC Normalize the Adipokine Profile in Adipose Tissue in an LTZ-Induced PCOS Mouse Model

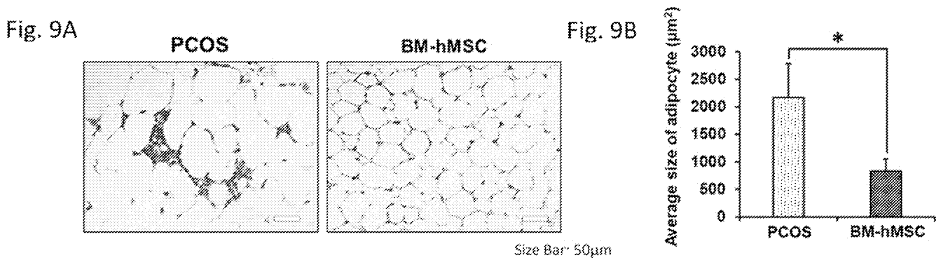

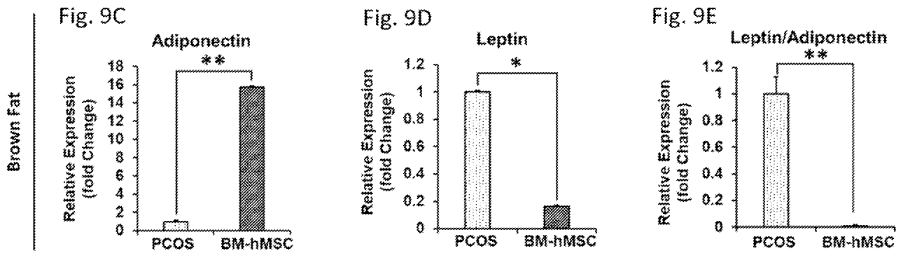

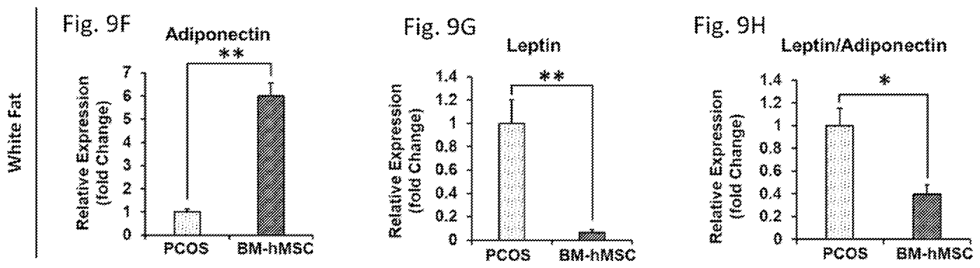

Weight gain associated with LTZ-induced PCOS is partially due to white fat expansion. The expansion of gonadal fat in the LTZ-induced PCOS mice was marked by characteristic morphologic enlargement of fat cells detected by H&E staining (FIG. 9A). Remarkably, the average size of adipocytes in the BM-hMSC-treated PCOS mice was significantly smaller than that in the untreated PCOS mice (FIG. 9B), approaching the normal size range of adipocytes.

White fat adipocyte expansion is usually associated with an increase in leptin that correlates inversely with adiponectin levels. Adiponectin is a pivotal adipokine that can reverse PCOS metabolism, acting as a humoral factor that regulates fat homeostasis by establishing cross-talk between white and brown fat cells. To explore such cross-talk in the PCOS mouse model, gene expression of leptin and adiponectin in brown adipose tissue as well as white gonadal fat was measured using qPCR. Treatment with BM-hMSC upregulated adiponectin and downregulated leptin expression, thus normalizing the ratio of leptin to adiponectin in brown fat tissue compared with the untreated group (FIGS. 9C-9E). Similar findings were observed in the white gonadal fat (FIGS. 9F-9H), highlighting the ability of BM-hMSC to normalize fat metabolism in the PCOS mouse model.

### Serum Hormone Analysis

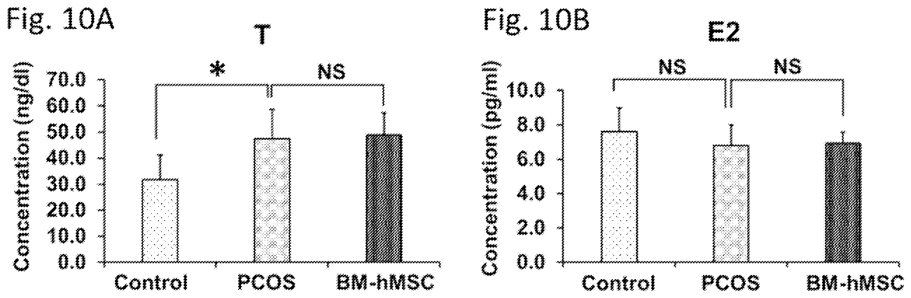

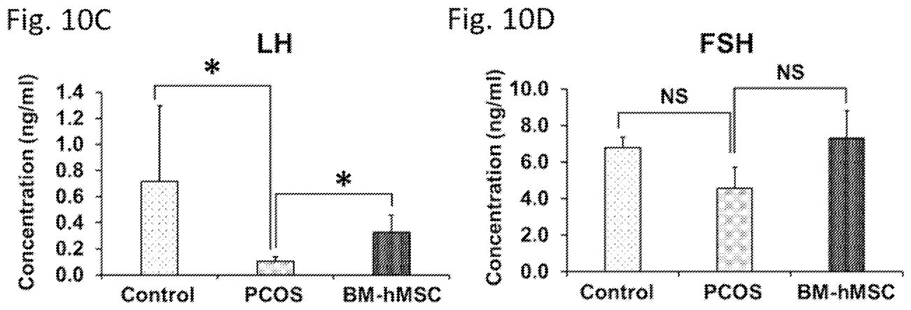

To assess the endocrine status following BM-hMSC engraftment, total serum hormone levels in BM-hMSC-treated and untreated PCOS animals were measured. Serum T levels were significantly higher in the untreated PCOS group versus healthy controls, with no significant difference in the BM-hMSC-treated group (p=0.797). Furthermore, there were no changes in serum estrogen levels among the three groups. However, LH was significantly lower in the PCOS group than healthy controls, and LH levels decreased after BM-hMSC engraftment in PCOS mice. In addition, FSH levels were lower in the PCOS group compared with healthy controls and increased after BM-hMSC treatment, though the change was not statistically significant (FIGS. 10A-10D).

### BM-hMSC Treatment Reverses Endometrial Abnormalities in an LTZ-Induced PCOS Mouse Model

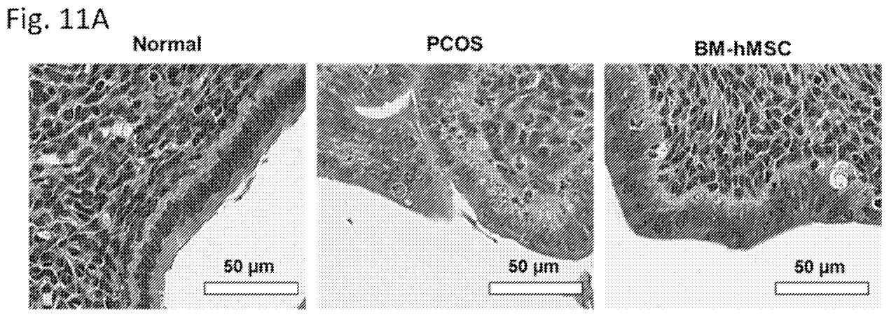

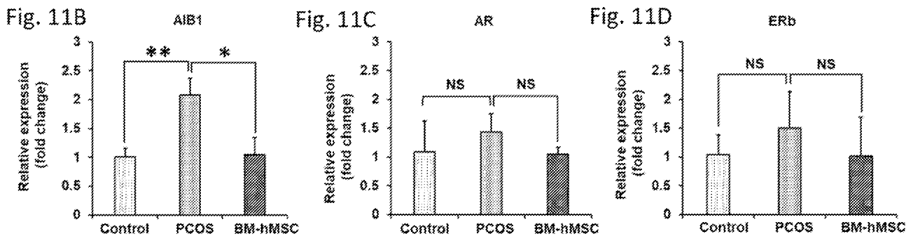

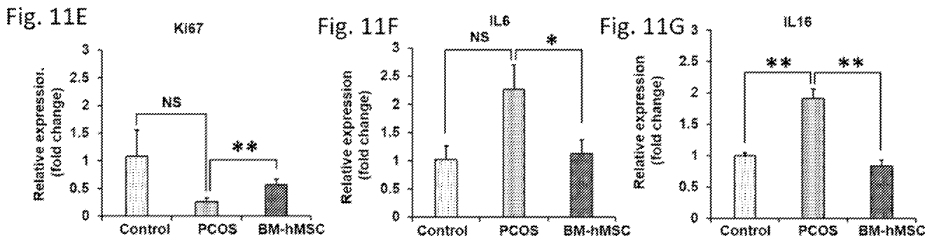

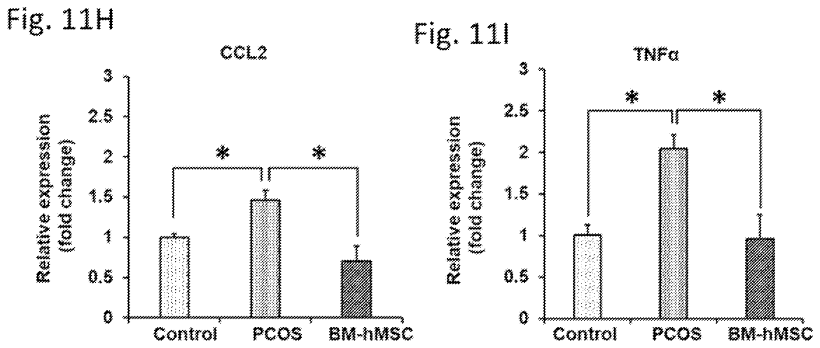

PCOS imparts abnormalities in endometrial tissue, such as the thickening of endometrium epithelial cells and aberration of steroid receptor gene expression. Consequently, endometrial tissue in BM-hMSC-treated versus untreated PCOS mice was analyzed. The endometrial tissue of the PCOS group showed abnormal thickness (FIG. 11A) and the AIB1 gene, known to be elevated in the PCOS endometrium, showed significant alterations in the PCOS group endometrium. These abnormalities were reversed in the BM-hMSC-treated PCOS group (FIG. 11B). Similarly, steroid receptor genes AR and ERP trended higher in the PCOS group and this was reversed after BM-hMSC treatment (FIG. 11C, 11D).

Interestingly, the proliferation marker K1-67 was significantly upregulated in BM-hMSC-treated PCOS mice compared with the untreated PCOS group (FIG. 11E). Additionally, several inflammatory regulator genes such as IL6, IL16, CCL2, and TNF-α were higher in the PCOS group endometrium compared with normal control endometrium, and these changes were significantly reversed after BM-hMSC treatment (FIGS. 11F-11I). These results suggest that intra-ovarian injection of BM-hMSC reversed various alterations in PCOS endometrium, at least partially, by normalizing steroid hormone receptors and inflammatory cytokine gene expression. These changes likely improved the quality of endometrium and contributed to the improved reproductive outcomes in the PCOS mice after BM-hMSC treatment.

### BM-hMSC Restore Fertility in an LTZ-Induced PCOS Mouse Model

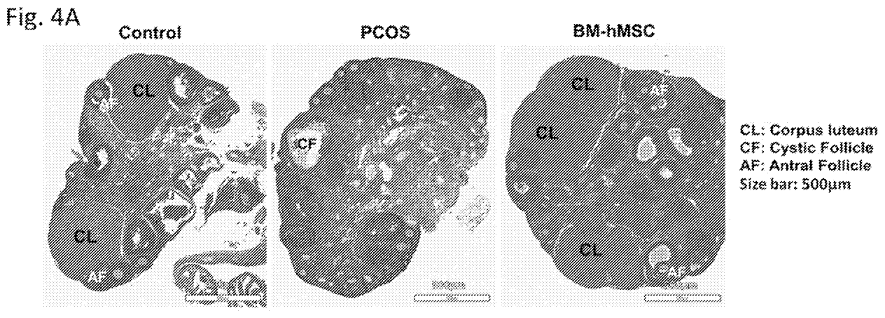

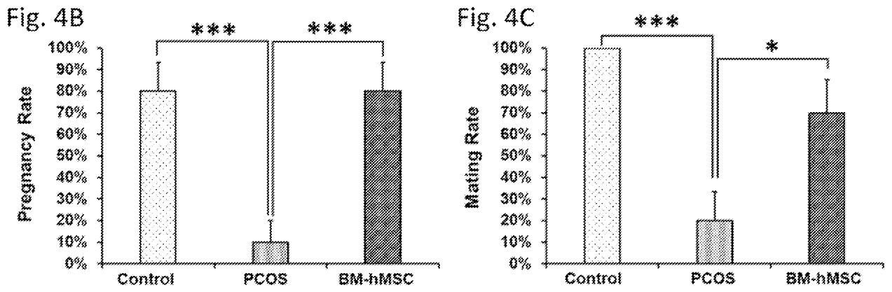

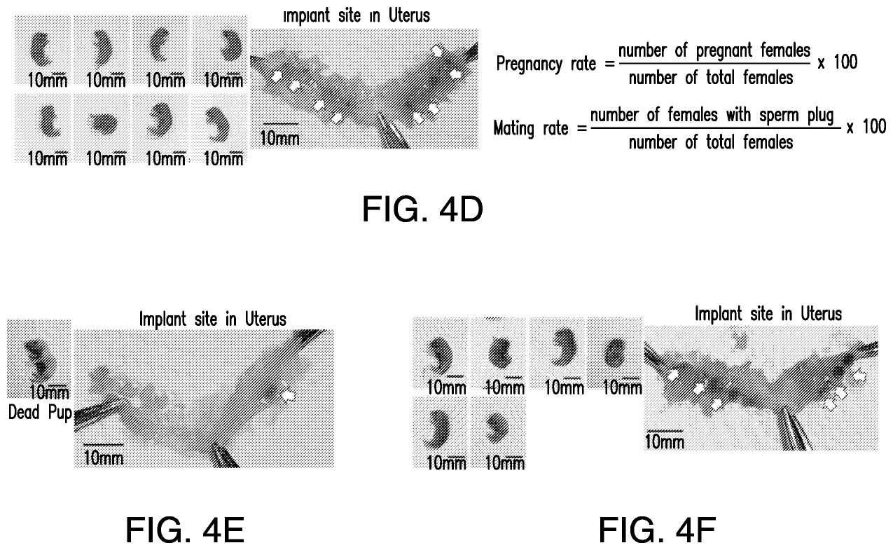

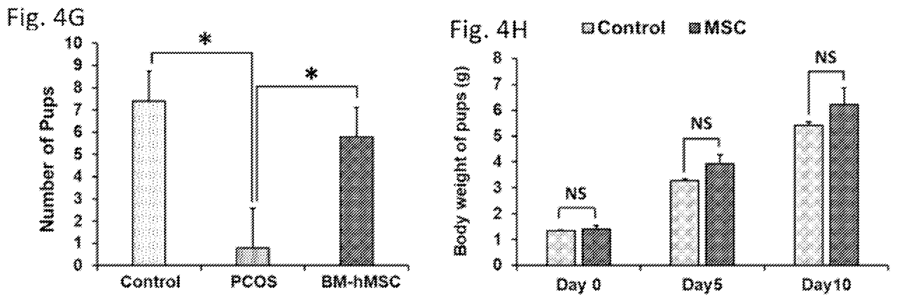

To explore the effect of BM-hMSC treatment on reproductive function, first analyzed was ovarian morphology in BM-hMSC-treated versus untreated PCOS mice. Ovaries from untreated PCOS mice displayed typical PCOS characteristics, including lack of corpora lutea and antral follicles compared with untreated normal control mice (FIG. 4A). After intra-ovarian engraftment of BM-hMSC in both ovaries of PCOS mice, normal ovarian morphology was partially restored, including the reappearance of corpora lutea and antral follicles, as well as well-ordered stroma that was morphologically similar to that of the normal control group. These morphological changes suggest that BM-hMSC engraftment improved the pathological changes in PCOS ovaries and may potentially restore ovulation in PCOS mice (FIG. 4A).

Next performed was a breeding experiment to test BM-hMSC's treatment capacity to restore fertility in the PCOS mouse model. It was found that healthy control mice had a higher rate of fertility (80%) than the subfertile PCOS group (10%). Interestingly, the pregnancy rate in BM-hMSC-treated PCOS mice was restored to a rate equal to that of the control group (FIG. 4B).

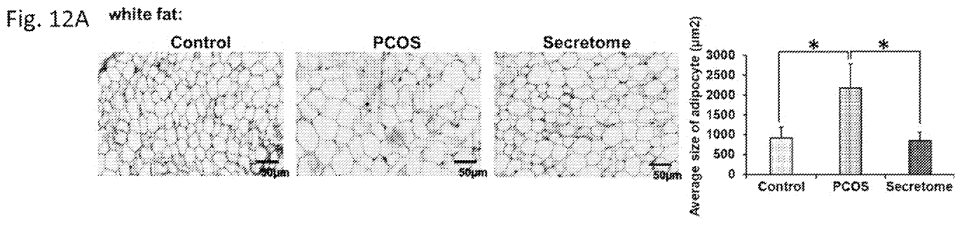

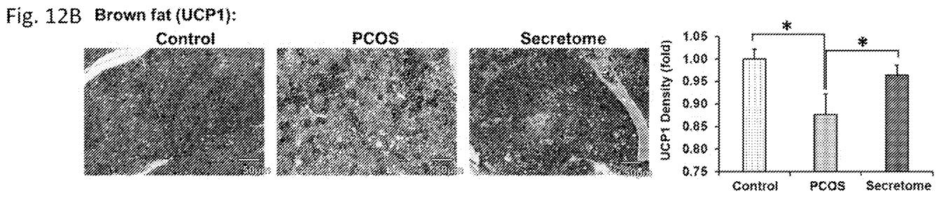

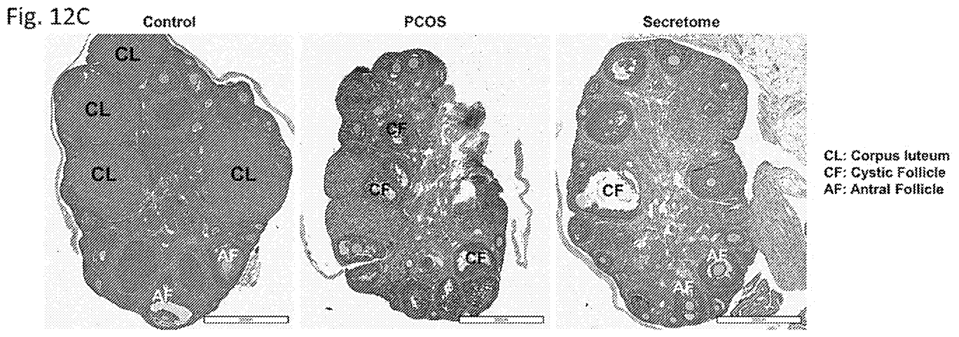

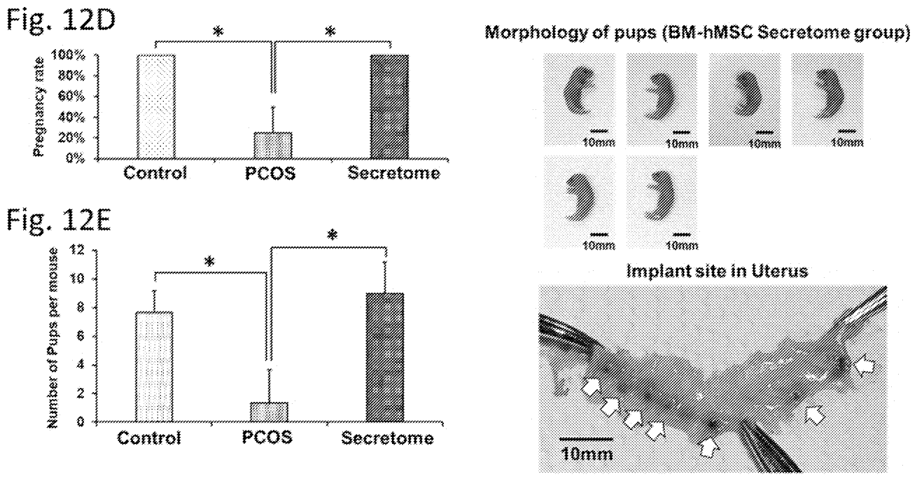

The number of delivered pups in all experimental groups was counted. As shown in FIGS. 4D-4G, it was found that most PCOS mice were infertile. The number of pups delivered in the BM-hMSC-treated PCOS group was equivalent to the number delivered in the control group. Moreover, the average number of pups delivered per mouse in the BM-hMSC-treated PCOS group (5.5±1.1) was significantly higher than that in the untreated PCOS group (0.8±1.7; FIG. 4G). Also found were no significant differences in the average body weight of the delivered pups between the control group and BM-hMSC-treated PCOS group at 0, 5, and 10 postnatal days (FIG. 4H). Notably, any apparent morphological abnormalities were not observed in any pups during the study period. The effect of injecting the BM-hMSC secretome in the PCOS mouse model was tested (FIG. 12). Fertility of PCOS mice was restored in secretome-treated animals. These results suggest that either BM-hMSC or its secretome can restore impaired fertility in an LTZ-induced PCOS mouse model with no detectable abnormalities in the delivered newborns.

### BM-hMSC Restore Normal Ovarian Gene Expression in an LTZ-Induced PCOS Mouse Model

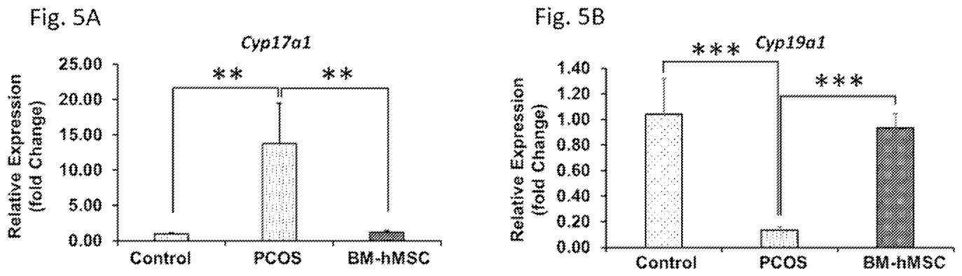

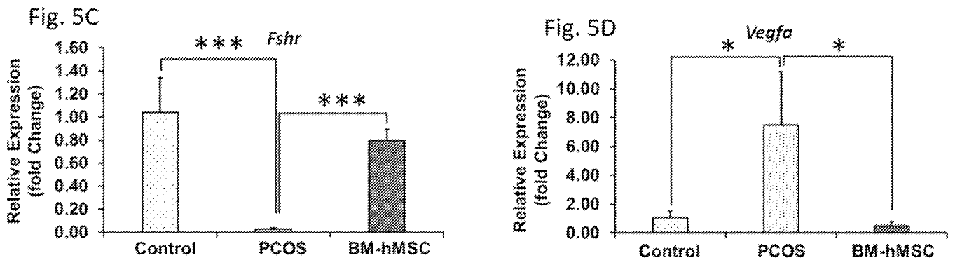

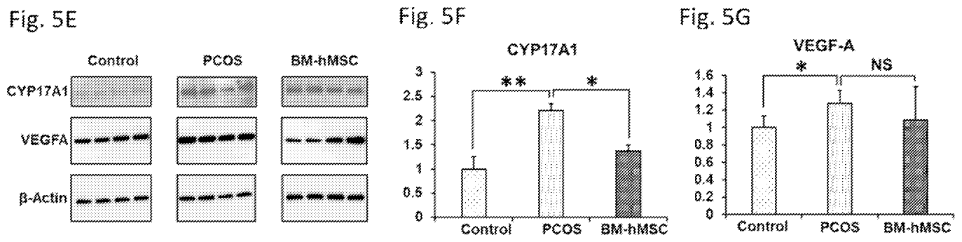

PCOS abnormalities include enhanced androgen production and altered ovarian angiogenesis. Next examined was the effect of BM-hMSC treatment on these abnormalities in the PCOS mice in vivo, to validate the in vitro data on the secretome effects on ovarian steroidogenic gene and inflammation marker expression. Mouse ovarian tissues from BM-hMSC-treated and untreated animals were analyzed for RNA and protein levels of steroidogenesis and angiogenesis markers. Cyp17a1 gene expression was significantly elevated in PCOS ovaries (13.73±5.78 fold), which was significantly reversed after BM-hMSC treatment (1.22±0.20 fold; FIG. 5A). Cyp19a1 (0.14±0.02 fold) and Fshr (0.03±0.01 fold) gene expression were lower in PCOS group ovaries, consistent with prior characterization of the LTZ-induced PCOS mouse model; levels of both genes significantly increased after BM-hMSC treatment (Cyp19a1: 0.93±0.11 fold, Fshr: 0.80±0.09 fold; FIGS. 5B, 5C). An abnormal increase in ovarian angiogenesis occurs in PCOS; thus, gene expression of angiogenesis marker Vegfa was measured in untreated and BM-hMSC-treated PCOS mice. Gene expression of Vegfa was elevated in the PCOS group (7.50±3.69 fold) and decreased after BM-hMSC treatment (0.48±0.29 fold; FIG. 5D). Immunoblot analysis supported these results, where CYP17A1 was higher in the PCOS group (2.21±0.14 fold) and significantly decreased after BM-hMSC treatment (1.37±0.12 fold; FIGS. 5E, 5F). Moreover, VEGF-A protein expression was elevated in PCOS mice (1.28±0.15 fold) and decreased after BM-hMSC treatment (1.08±0.38 fold), although the change was not statistically significant (FIGS. 5E, 5G). Taken together, the in vivo results suggest that BM-hMSC treatment can inhibit androgen synthesis and angiogenesis consistent with the effect of the BM-hMSC secretome on H295R cells.

### BM-hMSC Secretome Improves Metabolic and Reproductive Phenotypes in LTZ-Induced PCOS Mice

The in vitro and in vivo data suggest that the favorable effects of BM-hMSC engraftment likely occur in a paracrine fashion via secreted humoral factors in the BM-hMSC secretome. To explore the paracrine effect of BM-hMSC in the LTZ-induced PCOS mouse model, the BM-hMSC secretome was delivered by direct intra-ovarian injection into the ovaries of mice, and various metabolic and reproductive parameters were assessed. Analysis of white fat demonstrated a significant reduction in the size of fat cells in the secretome-treated PCOS group compared with the untreated PCOS group (FIG. 12A), and the expression of UCP1 in brown fat tissue was also significantly higher in the secretome-treated PCOS group compared with untreated PCOS group (FIG. 12B).

Morphological comparison of ovaries among the groups of mice by H&E staining revealed that secretome-treated PCOS ovaries had more antral follicles compared with untreated PCOS group ovaries (FIG. 12C). Importantly, secretome treatment also restored fertility in PCOS mice compared with the untreated PCOS control group (FIGS. 12D, 12E). These results confirm the ability of the BM-hMSC secretome to reverse metabolic and reproductive abnormalities in the LTZ-induced PCOS mouse model and suggest that the positive effects of BM-hMSC are primarily mediated via paracrine action of the BM-hMSC secretome.

### BM-hMSC Regulate Inflammation Via IL-10 in the LTZ-Induced PCOS Mouse Model

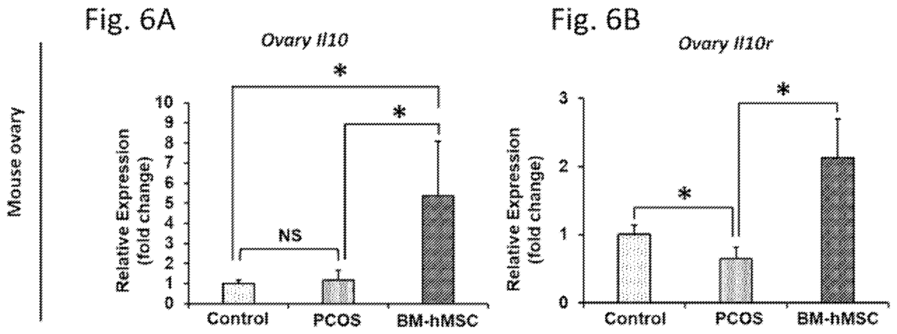

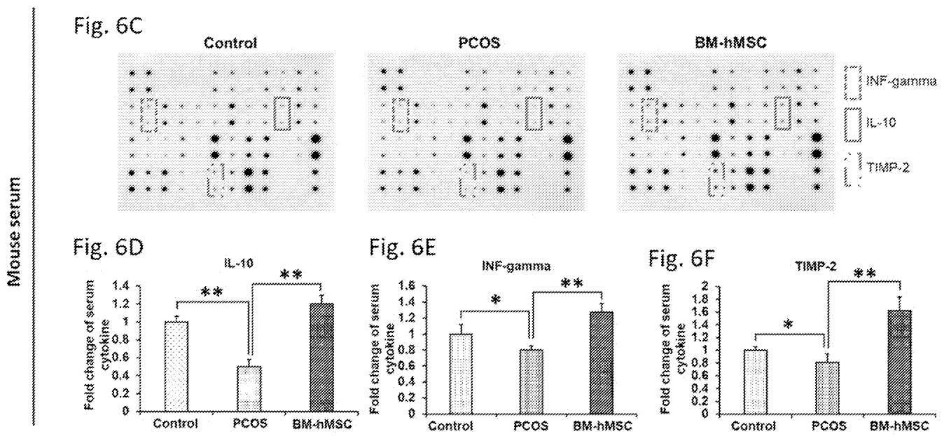

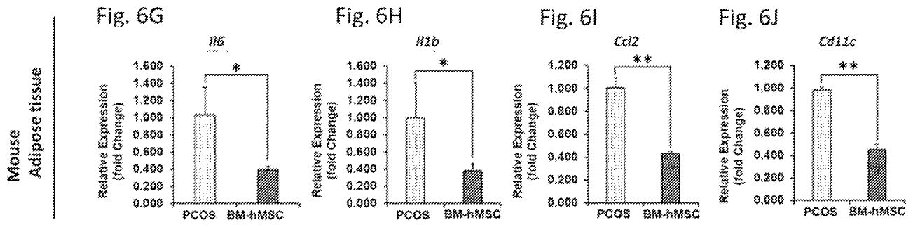

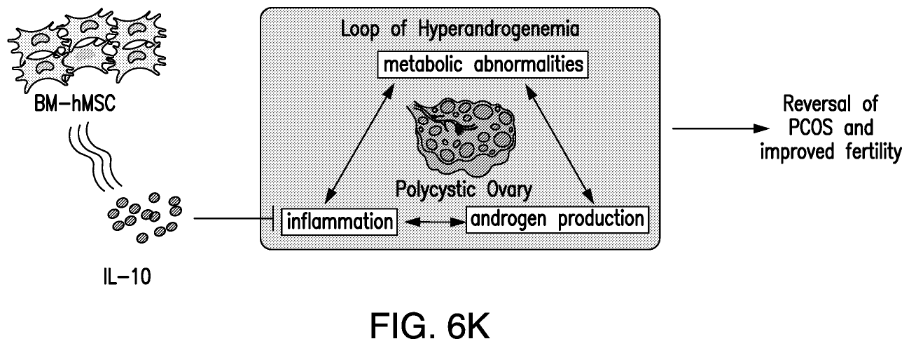

The data showed that BM-hMSC engraftment reverses several key PCOS-related features such as insulin resistance, increased expression of androgen synthesis genes, a pro-inflammatory milieu, and abrogated fat metabolism. Insulin resistance, androgen synthesis, and fat metabolism are all correlated with inflammation. Whether the effects of BM-hMSC treatment are mediated by anti-inflammatory factors within its secretome, such as IL-10, was explored. First analyzed was ovarian Il10 gene expression in all experimental groups. I110 gene expression in ovary tissue was significantly higher in BM-hMSC-treated PCOS ovaries (5.37±2.72 fold) compared with untreated PCOS ovaries (1.19±0.46 fold; FIG. 6A). Moreover, IL-10 receptor gene (Il10r) expression in ovary tissue was also significantly higher in the BM-hMSC-treated PCOS group (2.13±0.57 fold) compared with the untreated PCOS group (0.65±0.17 fold; FIG. 6B). An increased pro-inflammatory milieu in fat tissues occurs in PCOS women and animal models. The impact of intra-ovarian delivery of BM-hMSC on white gonadal fat tissue inflammatory markers was assessed using qPCR. BM-hMSC treatment significantly downregulated Il6 (0.39±0.04 fold), Il1b (1.0±0.41 fold), Ccl2 (0.43±0.02 fold), and Cd11c (0.45±0.05 fold) expression in the fat tissue of BM-hMSC-treated PCOS mice versus that of untreated PCOS mice (FIGS. 6G-6J). Several inflammatory regulators, such as IL-10, IFN-γ, and TIMP-2, have been found to be lower in PCOS patients compared with healthy women. Mouse serum was tested using an antibody-based membrane assay and found that these cytokines were significantly lower in the untreated PCOS group (IL-10: 0.50±0.08 fold, INF-γ: 0.79±0.05 fold, TIMP-2: 0.82±0.13 fold) compared with control mice (FIG. 6C). Importantly, these cytokines were significantly increased in PCOS mice after BM-hMSC treatment (IL-10: 1.20±0.09 fold, INF-γ: 1.27±0.11 fold, TIMP-2: 1.62±0.22 fold; FIGS. 6D-6F). These results suggest that intra-ovarian injection of BM-hMSC has a systemic anti-inflammatory effect in the PCOS mouse model, likely mediated by IL-10 secretion from these cells.

Taken together, the data suggests that intra-ovarian injection of BM-hMSC reduces inflammation by increasing the expression of anti-inflammatory mediators such as IL-10 and its receptor in the ovary, and circulating IL-10, IFN-γ, and TIMP-2 in serum, while decreasing pro-inflammatory mediators such as IL-6, IL-10, CCL2, and CD11c gene expression in periovarian adipose tissue.

### BMP-2 Measured in MSC Secretome

Bone morphogenetic proteins (BMPs) are among the many growth factors secreted by BM-hMSCs; these proteins play a key role in female fertility and are involved in all stages of folliculogenesis. A decrease in BMP levels has been indicated in PCOS in both animal models and patients. BMPs may play an important role in the pathogenesis of PCOS. Initially, the concentration of BMP-2 was quantified in the BM-hMSCs secretome by ELISA, using passages P3 to P5 BM-hMSCs (FIG. 13A). BMP-2 secretion was highest in P3 BM-hMSCs (150.8±1.8 pg/mL) but was not significantly different from P4 (125.8±1.8 pg/mL) and P5 (127.0±21.2 pg/mL).

### Effect of BMP-2 on H295R Cell Proliferation

Theca cell hyperplasia is a major finding in patients with PCOS and contributes to ovarian androgen oversecretion; therefore, the ability of BMP-2 to inhibit the proliferation of H295R cells in vitro was evaluated. Treatment with human recombinant BMP-2 significantly decreased H295R cell proliferation in a dose- and time-dependent manner (FIG. 13B-D). In the cell counting experiment, 24 h after treatment, untreated H295R cells (control) numbered 1.53±0.04×10⁵, while BMP-2 treated-cells showed decreased cell numbers (3.125 ng/mL: 1.47±0.04×10⁵, 6.25 ng/mL: 1.39±0.03×10⁵, 12.5 ng/mL: 1.36±0.03×10⁵, 25 ng/mL: 1.29 0.01×10⁵, 50 ng/mL: 1.23 0.03×10⁵, 100 ng/mL: 1.15 0.06×10⁵) compared to untreated H295R cells. After 72 h, BMP-2 treated-cells showed significantly decreased cell numbers (3.125 ng/mL: 1.71±0.03×10⁵, 6.25 ng/mL: 1.23±0.05×10⁵, 12.5 ng/mL: 1.20 0.04×10⁵, 25 ng/mL: 1.14 0.03×10⁵, 50 ng/mL: 1.17 0.03×10⁵, 100 ng/mL: 1.11±0.02×10⁵) while untreated control H295R cells were proliferating well (2.05±0.03×10⁵). One hundred and twenty hours after treatment, the number of untreated control H295R cells was 2.26±0.08×10⁵ and that of all of the BMP-2 treated-cells showed a dose-dependently decreased number (3.125 ng/mL: 2.0±0.06×10⁵, 6.25 ng/mL: 1.38±0.06×10⁵, 12.5 ng/mL: 1.18±0.09×10⁵, 25 ng/mL: 1.12±0.07×10⁵, 50 ng/mL: 1.12±0.02×10⁵, 100 ng/mL: 1.07 0.01×10⁵).

### Effect of BMP-2 on PCOS-Related Parameters in H295R Cells

Next, the effect of BMP-2 on various PCOS-related parameters in H295R cells was assessed. As a first step, androgen synthesis by H295R cells was analyzed. The RT-PCR results (FIG. 13E-G) show that BMP-2 treatment significantly downregulated the expression of two key androgen-synthesizing enzymes relative to their expression levels in a control cell set at 1.0, CYP17A1 (3.125 ng/mL: 0.63±0.04-fold, 6.25 ng/mL: 0.39±0.03-fold, 12.5 ng/mL: 0.70±0.04-fold, 25 ng/mL: 0.49±0.05-fold) and DENND1A (3.125 ng/mL: 0.54±0.11-fold, 6.25 ng/mL: 0.50±0.04-fold, 12.5 ng/mL: 0.69±0.07-fold, 25 ng/mL: 0.66±0.01-fold), but had no significant effect on CYP11A1 gene expression (3.125 ng/mL: 1.19±0.11-fold, 6.25 ng/mL: 0.99±0.11-fold, 12.5 ng/mL: 1.57±0.04-fold, 25 ng/mL: 1.04±0.01-fold). The gene expression levels were analyzed using RT-PCR and normalized by the GAPDH gene.

Effect of BMP-2 on Ovarian Granulosa Cell (hGrC1)

Granulosa or follicular cells are somatic cells whose major functions include production of steroidal hormones and growth factors involved in oocyte development. Granulosa cells are an in vitro model of POI, and some studies also use them as a PCOS model. The effect of BMP-2 on the expression of estrogen-producing genes Cyp19 and StAR was analyzed. The gene expression levels were not consistent for the dose of BMP2 (FIG. 13H-I). Only the specific BMP-2 concentration 25 ng/ml showed significant enhancement of Cyp19 and StAR gene expression.

Taken together, these results indicate that BMP-2 is a key player mediating the favorable effects of the BM-hMSCs secretome in a human PCOS cell model. BMP-2 overexpression could increase the efficacy of BM-hMSC-based therapy, serving as a novel stem cell therapy for patients with intractable PCOS.

### TGFβ-1 Measured in MSC Secretome

Studies have shown that TGFβ-1 is an anti-inflammatory cytokine secreted by MSCs. The concentration of TGFβ-1 was measured in unmodified MSC secretomes. The level of TGFβ-1 in human dermal fibroblast conditioned medium (HDF CM) was used as a control (FIG. 14A). The level of TGFβ-1 was: in total MSC HDF-CM, 269.5±9.192 pg/ml; in total concentrated MSC HDF-CM after 24 hrs, 719.5±23.33 pg/ml; in total MSC HDF-CM after 48 hrs, 866±22.63 pg/ml; in total concentrated MSC HDF-CM after 48 hrs, 988.0±41.1 pg/ml; with the level of TGFβ-1 in the concentrated HDF-CM control being 301.0±4.243 pg/ml.

### Effect of TGFβ-1 Treatment on In Vitro PCOS Model Androgen Producing Cells (H295R)

Next, the ability of TGFβ-1 treatment to suppress androgen producing gene expression (Cyp17A1, Cyp11A1, DENND1A) in was determined in H295R cells. The gene expression levels were analyzed using RT-PCR and normalized by GAPDH gene. The RT-PCR results (FIG. 14C-D) show that TGF β-1 treatment significantly downregulated the expression of two key androgen-synthesizing enzymes, CYP17A1 (3.125 ng/mL: 0.37±0.03-fold, 6.25 ng/mL: 026±0.01-fold, 12.5 ng/mL: 0.15±0.03-fold, 25 ng/mL: 0.25±0.02-fold) and DENND1A (3.125 ng/mL: 0.22±0.1-fold, 6.25 ng/mL: 0.14±0.02-fold, 12.5 ng/mL: 0.06±0.01-fold, 25 ng/mL: 0.07±0.01-fold), but had no significant effect on CYP11A1 gene expression (3.125 ng/mL: 0.59±0.7-fold, 6.25 ng/mL: 05.25±7.27-fold, 12.5 ng/mL: 3.57±3.15-fold, 25 ng/mL: 2.1±0.33-fold).

### Effect of TGFβ-1 Treatment on In Vitro PCOS Model Androgen Producing Cells (H295R).

Next, the ability of TGFβ-1 treatment to suppress the testosterone level in H295R cells grown in HDF-CM was studied. Total testosterone level were measured by ELISA. The results (FIG. 14E) demonstrated significant suppression of testerone production at all TGFβ-1 concentrations tested—control with no TGFβ-1: 2.110±0.3516, 3.125 ng/mL: 0.458±0.108 ng/ml, 6.25 ng/mL: 0287±0.264 ng/ml, 12.5 ng/mL: 0.403±0.070 ng/ml, 25 ng/mL: 0.387±0.100 ng/ml.

Taken together, these results indicate that TGFβ-1 is a key player mediating the favorable effects of the BM-hMSCs secretome in a human POI cell model. TGFβ-1 overexpression could increase the efficacy of BM-hMSC-based therapy, serving as a novel stem cell therapy for patients with POI.

### DISCUSSION

A significant inhibitory effect of the BM-hMSC secretome was observed on steroidogenesis gene expression, inflammation, and androgen production in H295R cells, as well as in primary cultures of theca cells from women with PCOS. Additionally, the in vivo experimental data showed that intra-ovarian engraftment of BM-hMSC is capable of correcting several PCOS-related metabolic abnormalities in a mouse model of PCOS. While this LTZ-induced PCOS mouse model is infertile, it was demonstrated that BM-hMSC treatment was able to restore fertility and treated mice delivered healthy pups. Interestingly, similar improvements in metabolic and reproductive endpoints were achieved with injection of BM-hMSC secretome, suggesting that most, if not all, of BM-hMSC effects in this model are paracrine in nature.

Chronic inflammation plays an important role in PCOS pathogenesis. BM-hMSC engraftment significantly reduced several inflammatory markers in PCOS mouse ovaries. A positive feedback loop exists between inflammation and androgen production, suggesting that androgen synthesis and inflammation could be reciprocally self-propagated. Up-regulation of CYP17A1 gene expression through oxidative stress, which is a known stimulator of inflammation, also demonstrates a positive feedback loop in PCOS. While there was significant suppression of androgen production in vitro after BM-hMSC secretome treatment, no difference was observed in serum testosterone levels between the untreated PCOS and BM-hMSC-treated PCOS groups. This could be attributed to the episodic nature of steroid hormone secretion. Furthermore, the findings may highlight a limitation of the chemically (LTZ)-induced PCOS model, which primarily relies on the induction of higher testosterone accumulation via marked supraphysiological inhibition of its aromatization. Key ovarian steroidogenic genes as Cyp17a1 were upregulated in the PCOS group, and significantly suppressed by BM-hMSC treatment. The effect of engrafted BM-hMSC on ovarian cells could occur via cell-to-cell contact or paracrine effects through secreted humoral factors.

IL-10 is an important immune-suppressive and anti-inflammatory cytokine that is key to several human disorders, including PCOS. Significantly lower serum levels of IL-10 occur in PCOS women compared with age- and BMI-matched healthy controls. BM-hMSC secrete physiologically relevant quantities of IL-10, which is confirmed in the BM-hMSC here (FIG. 2A). It was shown that IL-10 treatment significantly downregulates steroidogenesis and inflammatory gene expression as well as suppresses androgen production by H295R cells (FIG. 2F). In vivo, BM-hMSC treatment significantly increased IL-10 expression in ovarian tissue and its serum concentration in PCOS mice. These results suggest that BM-hMSC can ameliorate PCOS-induced inflammation through IL-10 secretion, and IL-10 overexpressing-BM-hMSC might be a novel and robust therapeutic approach for PCOS treatment (FIG. 6K). Similar findings for factors BMP-2 and TGFβ-1 suggest that BM-hMSC overexpressing BMP-2 or TGFβ-1 also might be an effective therapeutic approach for PCOS or POI treatment.

### Example 2

Overexpression of miR144-5p Enhances Regenerative Capacity of Human Mesenchymal Stem Cells in a Chemotherapy-Induced Premature Ovarian Insufficiency Cell Model.

miR144-5p-overexpressing MSCs were established with lentivirus transfection. MSCs were seeded in complete medium into multiwell plates 24 hours prior to transfection. After transfection was completed, cells were analyzed based on RT-PCR results and fluorescence (GFP) expression. The effect of condition media and purified exosome from miR144 overexpressing MSCs on granulosa cells was studied.

MSC-miR144 condition media was used to treat chemotherapy (CTX) treated HGrC1. After treating cyclophosphamide-treated HGrC1 with condition media for 24 hours, cell proliferation and steroidogenesis gene expression were analyzed. Cell proliferation was detected by cell count, and it was significantly higher (1.478±0.36-fold, p<0.05) than the unenhanced MSC condition media (CM) treated group (1.01±0.265-fold, p<0.05). The expression of steroidogenesis genes such as CYP19A1 (505.74±52-fold p<0.05) and StAR (34.31±7.3-fold, p<0.05) were significantly increased in RT-PCR results compared to the unenhanced MSCs CM group. See FIG. 15A.

Next, similar experiments were performed comparing the results of treatment with MSC-derived secretome from control MSC with those from treatment with secretomes derived from three clones of MSC overexpressing miR144 (FIG. 15B-15D, miR144-MSCCM #1 to #3). Human granulosa cells (hGrC1) were treated with the MSC CM secertomes. Granulosa cells are an in vitro model of POI, but some studies have used this type of cell as a model of PCOS. The effect of miR144 overexpressing-MSC on hGrC1 cell proliferation (FIG. 15B), as well as on expression levels of estrogen producing genes (Cyp19 and StAR; FIGS. 15C and D, respectively) was analyzed.

At least 2/3 of the clones (#2 and #3) enhanced proliferation of hGrC1 1.5±0.4 fold (FIG. 15B, control MSC-CM: 1.011±0.265, #1: 0.925±0.225, #2: 1.457±0.355, #3: 1.478±0.360). Similarly, clones #2 and #3 also enhanced the gene expression of estrogen producing-gene Cyp19a1 (Cyp19) in CM treated-hGrC1 at least ˜100-fold (FIG. 15C, control MSC-CM: 3.09±4.04, #1: 20.8±17.4, #2: 293.0±79.9, #3: 505.7±51.9). The 3 miR144 overexpressing-MSC clones enhanced the gene expression of estrogen producing gene StAR (Steroidogenic Acute Regulatory protein-encoding gene) in CM-treated hGrC1 an average of 18-fold (FIG. 15D, control MSC-CM: 1.64±1.95, #1: 34.31±7.30, #2: 27.58±0.35, #3: 28.54±0.19). The gene expression levels were analyzed using RT-PCR and normalized by the GAPDH gene.

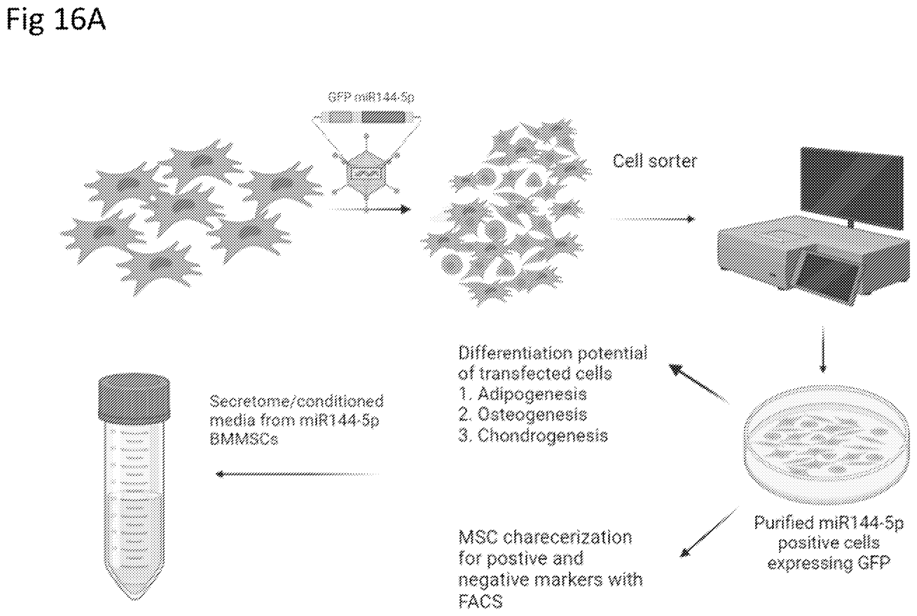

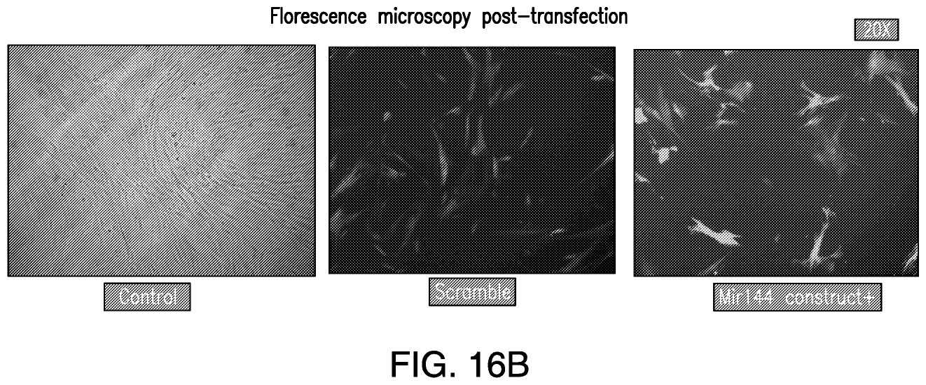

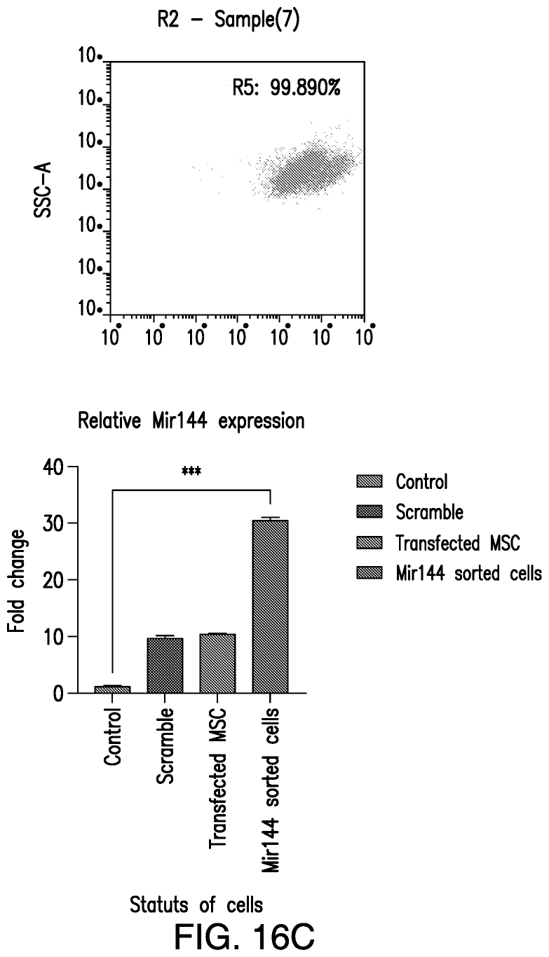

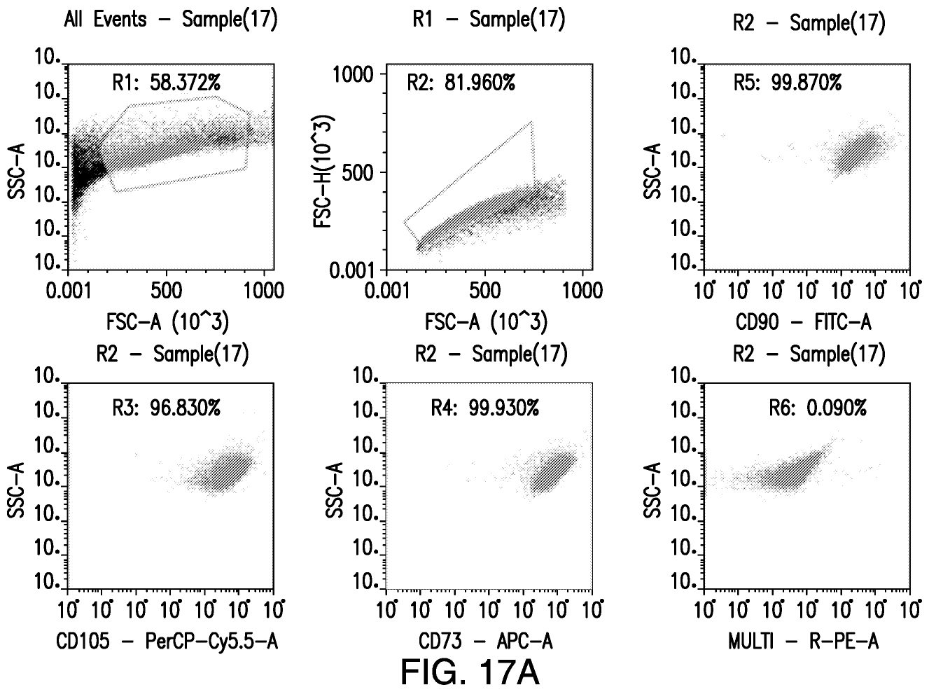

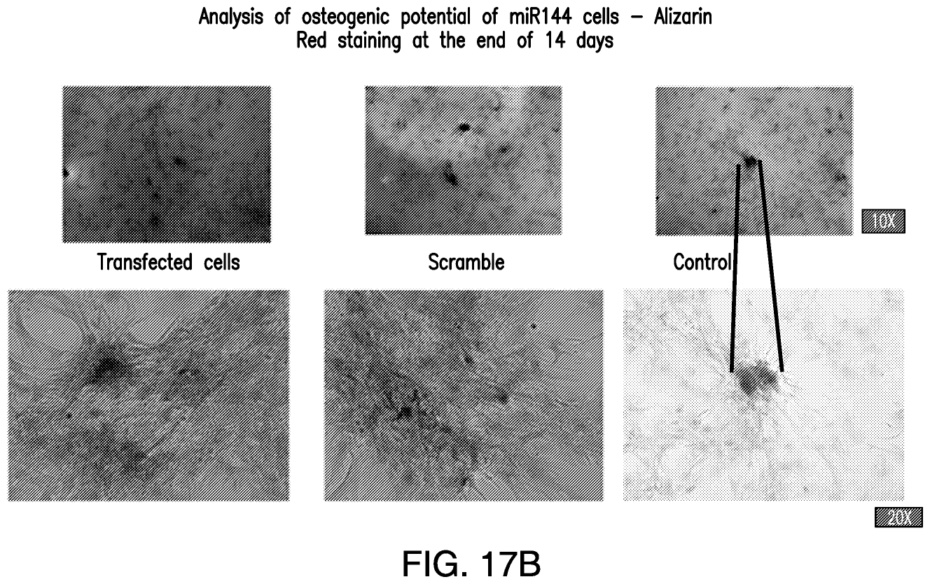

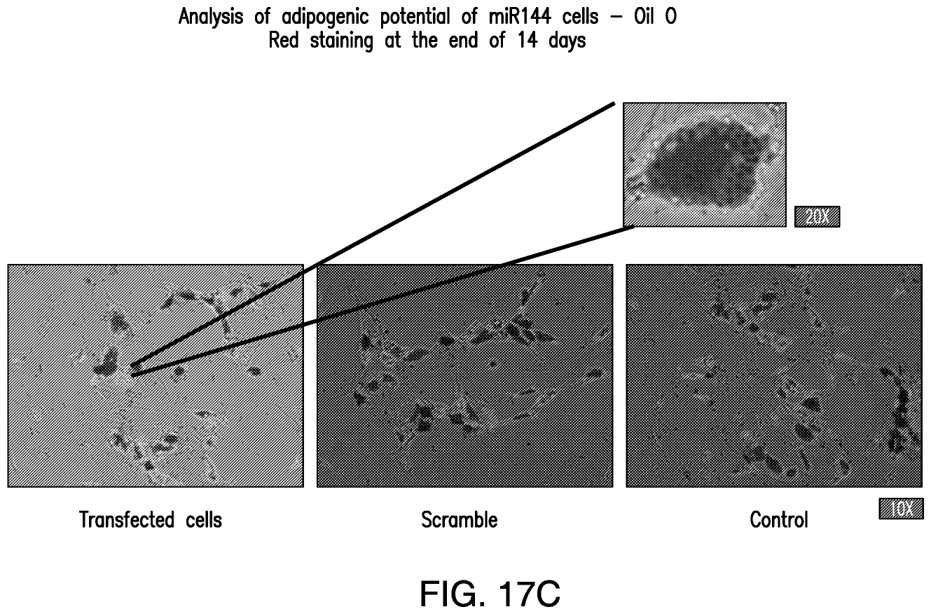

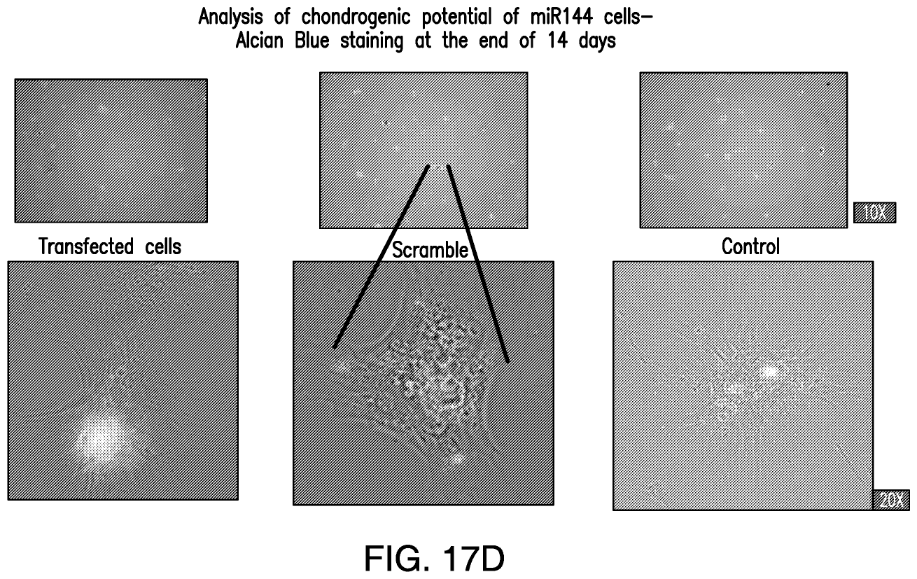

Through lentivirus mediated transfection and cell sorting, miR144-overexpressing MSCs were purified from mixed population. The purified miR144-overexpressing cells were showing 99.89% purity and expressing miR144 around 30-fold higher compared to healthy control MSC, and approximately 3-fold higher compared to mixed population cells (FIG. 16C). These miR144-overexpressing cells still express MSC specific surface markers such as CD90 (99.87%), CD105 (96.83%), CD73 (99.93%), and do not express the negative markers such as CD45, CD34, CD11b, CD19 and PE HLA-DR (0.090%). Three lineage differentiation potential was confirmed after osteogenic differentiation with Alizarin Red staining (FIG. 17B), adipogenic differentiation with Oil O Red staining (FIG. 17C), and chondrogenic differentiation with Alcian blue staining (FIG. 17D).

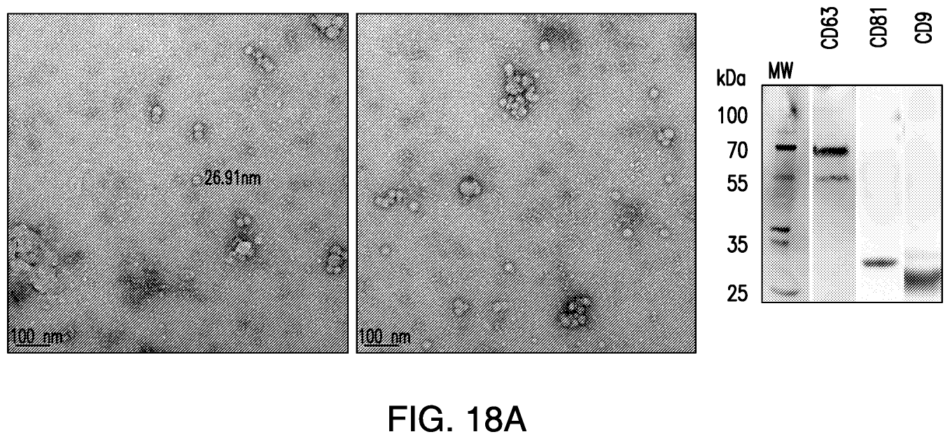

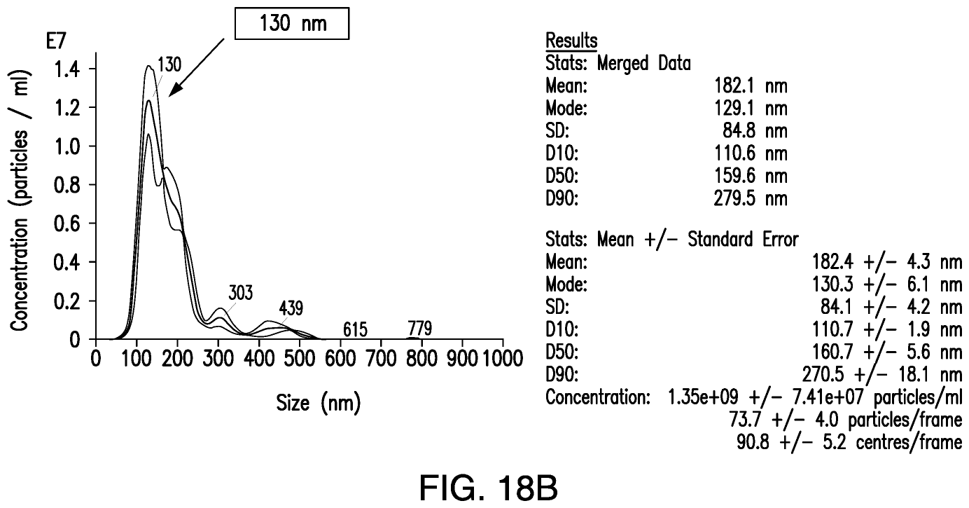

Exosomes were isolated from miR144-overexpressing MSC using a standard protocol (precipitation or ultracentrifuge). The general structure of exosomal vesicle was observed using electron microscopy (TEM). It was confirmed that the miR144-overexpressing exosomes are expressing exosome specific marker proteins such as CD63, CD81, and CD9 (FIG. 18A). NTA analysis data shows that the average size of exosomes are around 129.1±84.8 nm and the concentration was approximately 1.35×10⁹ particles/ml (FIG. 18B).

The therapeutic effect of miR144-overexpressing exosomes was tested using an in vitro POI model of damaged human granulosa cells (hGrC1). Healthy hGrC1 were treated with 200 μg/ml cyclophosphamide to make damaged hGrC1. Cell counting data indicated exosome-treated damaged cells showed increased cell number compared to untreated damaged granulosa cells and was almost equal to healthy cell control. The viability (percentage of live cells) also significantly increased in exosome treated cells compared to untreated damaged cells.

### Example 3

Co-Culture with Target Cells Enhances Regenerative Capacity of Human Mesenchymal Stem Cells in Chemotherapy-Induced Premature Ovarian Insufficiency Cell Model.

Healthy hGrC1 (H-GCs) were treated with 200 μg/ml cyclophosphamide to make sick hGrC1 (S-GCs). To establish co-culture conditions, a transwell system was used. MSCs were cultured alone (MSCs), with S-GCs (MSCs/S-GCs), or with H-GCs (MSCs/H-GCs) to produce enhanced MSC secretome. Twenty-four hours after secretome therapy on damaged granulosa cell (S-GCs), cell proliferation, viability, apoptosis, and steroidogenic marker expression were compared between samples.

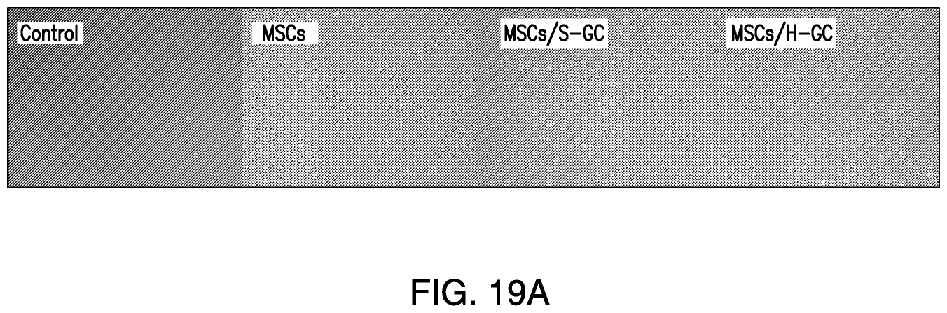

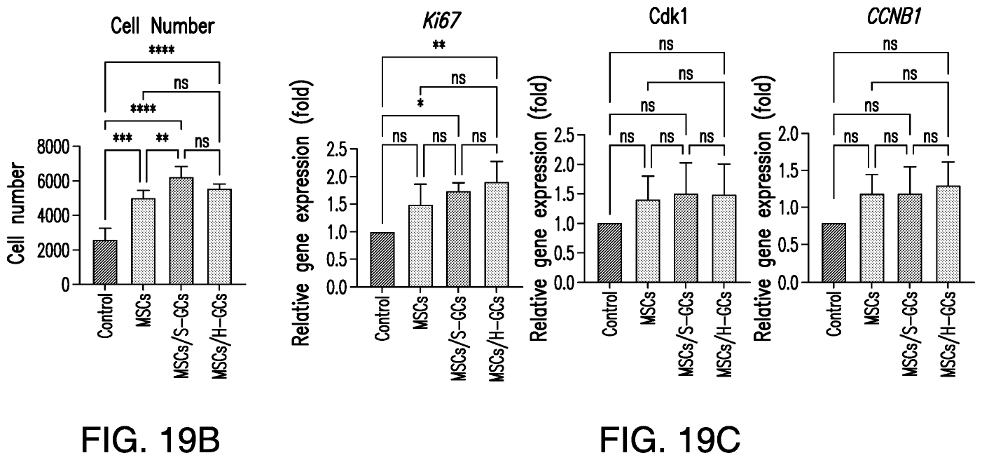

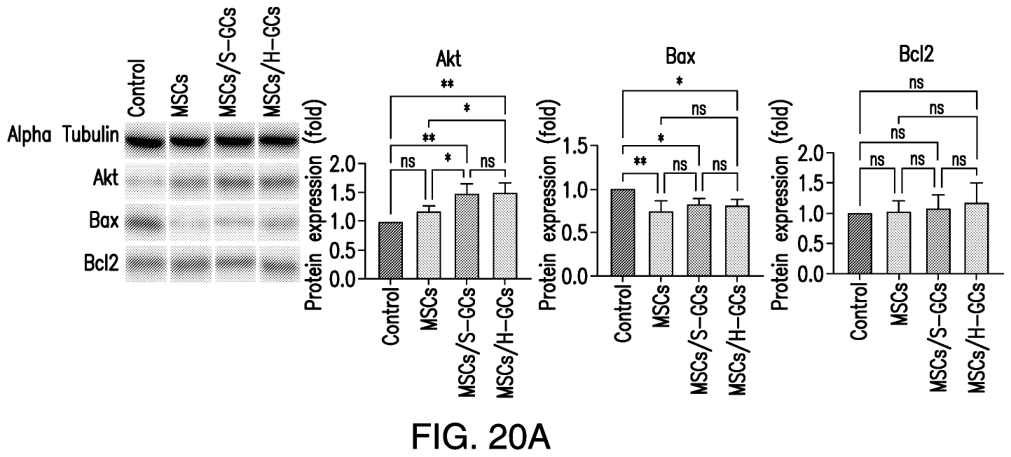

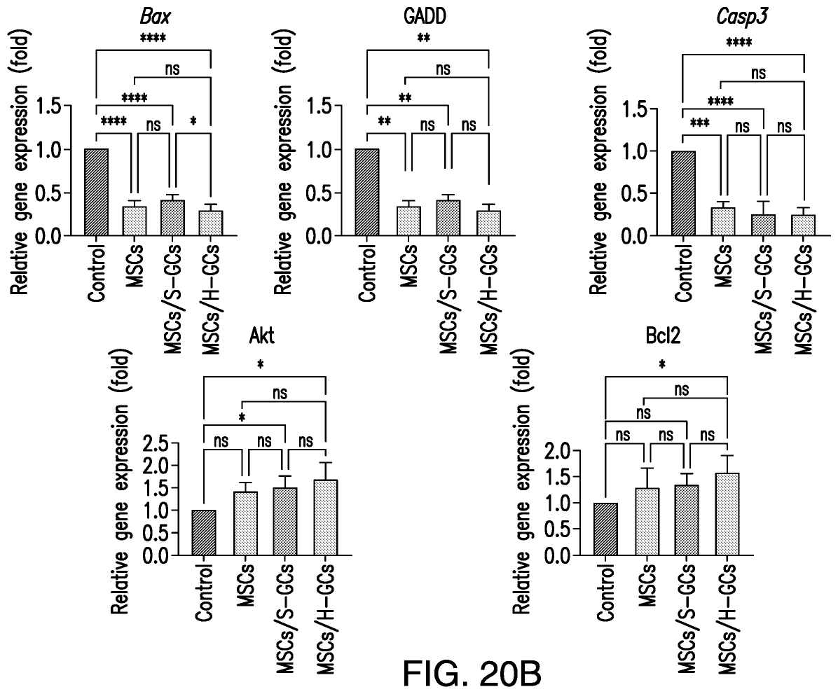

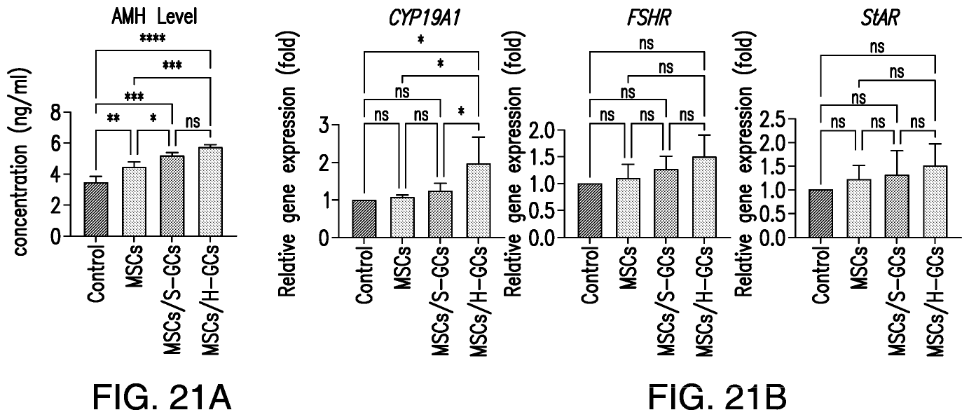

Cell viability and cell number were successfully increased in S-GC treated by secretome purified from MSC/S-GS co-culture and MSC/H-GS co-culture. Secretome purified from MSC/S-GS co-culture and MSC/H-GS co-culture also increased the levels of expression Ki67, cdk1, and CCNB1 in S-GC. See FIGS. 19A-C. In apoptosis marker analysis, MSC/S-GC co-culture secretome and MSC/H-GC co-culture secretome therapy significantly increased the Akt protein expression (anti-apoptotic protein) as well as significantly reduced Bax protein expression (proapoptotic protein) in sick GC (FIG. 20A). MSC/S-GC co-culture secretome and MSC/H-GC co-culture secretome therapy increased the expression of Akt and Bcl2 genes (anti-apoptotic protein) as well as significantly decreased the expression of Bax, GADD, and Casp3 (proapoptotic protein) in S-GC (FIG. 20B). The AMH levels significantly increased in all three treated groups than control (FIG. 21A). The effect of MSC secretome, MSC/S-GC co-culture secretome, and MSC/H-GC co-culture secretome treatment on hGrC1 marker genes was evaluated by gene expression analysis of estrogen-producing genes Cyp19A1, StAR, and FSHR. The expression of StAR and FSHR increased in S-GC treated by MSC/H-GC co-culture secretome, and the expression of estrogen-producing genes Cyp19A1 significantly increased in S-GC treated by MSC/H-GC co-culture secretome (FIG. 21B).

This study demonstrated the potential of a co-culture system to produce enhanced secretion factors, including engineered exosome, for effective treatment for damaged ovary.

### Example 4

### Prevention of POI and Chemotherapy-Induced Damage by Exosome Treatment in Mouse Models.

C57/BL6 mice were used for the animal model. MSC-derived exosomes (1.0 to 1.5×10⁸ exosomal particles/10 uL) were directly injected into each ovary by laparotomy. After laparotomy, chemotherapy reagent cyclophosphamide (120 mg/kg) and busulfan (30 mg/kg) were injected via intraperitoneal route to induce ovary damage and POI condition. One week after chemotherapy, mice were mated with male mouse for breeding experiment. Approximately 3 weeks after mating, the fertility was analyzed by percentage of pregnant mice and number of delivered pups.

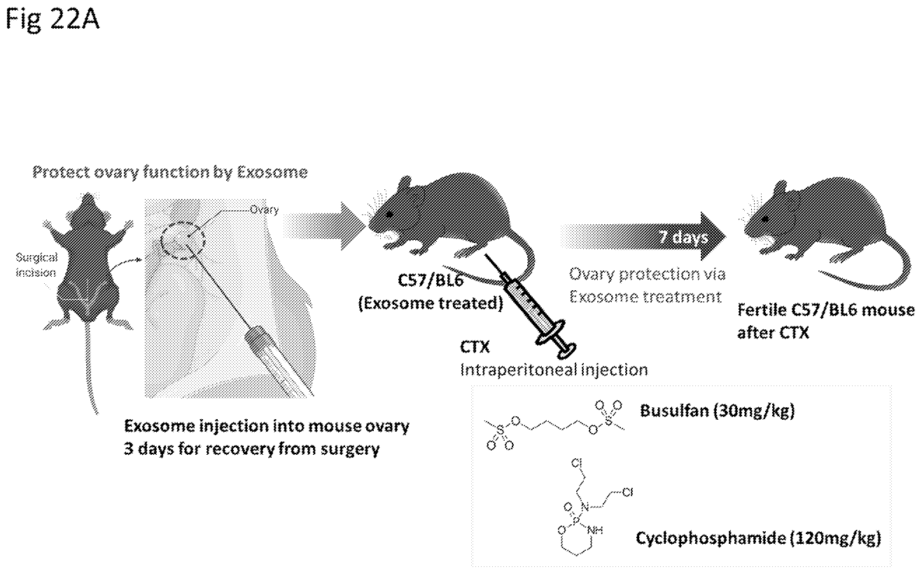

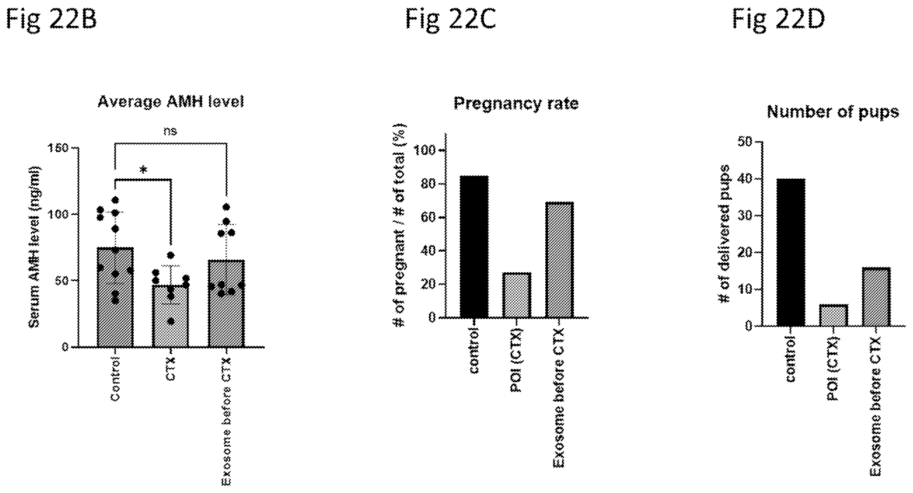

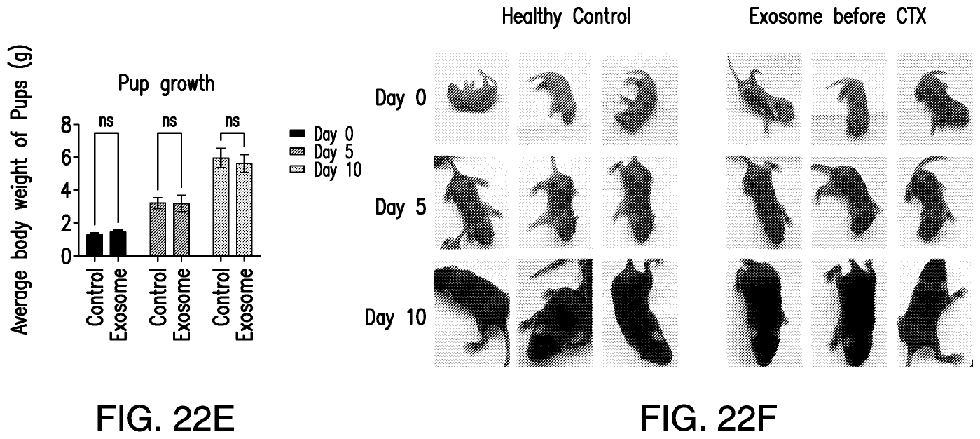

The prevention effect on chemotherapy-induced damage by of exosome treatment were analyzed by breeding experiment using mouse model. Exosome were administered prior to the chemotherapy, which induces the POI condition (FIG. 22A). The exosome-treated mice were still fertile after chemotherapy, while untreated mice became infertile after chemotherapy (FIG. 22 B-D). Offspring delivered from exosome-treated mice gre well and did not show significant difference compared to healthy mice to postnatal 10 days (FIGS. 22 E-22F).

These studies demonstrate the exosome treatment can prevent ovary damage induced by chemotherapy and maintain fertility after chemotherapy in animal model.

### Example 5

### Effects of Exosomes on Male Fertility.

Chemotherapy reagents kill cells by inducing DNA damage or oxidative stress, and this mechanism is the same even in different types of cells, including the cells of the ovaries in female mammals and the cells of the testes in male mammals.

A male mammal is treated with MSC-derived exosomes prior to chemotherapy. The cells of the testes are protected from chemotherapy-induced damage.

A pre-pubescent male mammal is treated with MSC-derived exosomes prior to chemotherapy. The cells of the testes are protected from chemotherapy-induced damage.

A human male is treated with MSC-derived exosomes prior to chemotherapy. The cells of the testes are protected from chemotherapy-induced damage.

A pre-pubescent human male is treated with MSC-derived exosomes prior to chemotherapy. The cells of the testes are protected from chemotherapy-induced damage.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing premature ovarian insufficiency (POI) or polycystic ovary syndrome (PCOS) in a female mammal, the method comprising administering to the female mammal an effective amount of (1) mesenchymal stem cells (MSCs), (2) secretome from MSCs, (3) exosomes produced by MSCs, or (4) exosomes comprising one or more effectors, wherein the MSCs overexpress (a) miR144, (b) BMP-2, (c) TGFβ1, (d) IL-10, or (e) any combination of (a), (b), (c) and (d), wherein the one or more effectors comprises (i) miR144, (ii) BMP-2, (iii) TGFβ1, (iv) IL-10, or (v) any combination of (i), (ii), (iii) and (iv), and wherein the amount of the effector per exosome is greater than the amount of the effector per exosome when produced by unmodified mesenchymal stem cells (MSCs).

2. The method of claim 1, wherein the method comprises administering MSCs.

3. The method of claim 1, wherein the method comprises administering secretome from MSCs.

4. The method of claim 1, wherein the method comprises administering exosomes produced by MSCs.

5.-8. (canceled)

9. The method of claim 1, wherein the method comprises administering exosomes comprising one or more effectors.

10. The method of claim 1, wherein the method comprises administering the (1) MSCs, (2) secretome from MSCs, or (3) exosomes produced by MSCs, wherein the MSCs overexpress miR144, or (4) exosomes comprising one or more effectors, wherein the one or more effectors comprises miR144.

11. The method of claim 1, wherein the method comprises administering the (1) MSCs, (2) secretome from MSCs, or (3) exosomes produced by MSCs, wherein the MSCs overexpress BMP-2, or (4) exosomes comprising one or more effectors, wherein the one or more effectors comprises BMP-2.

12. The method of claim 1, wherein the method comprises administering the (1) MSCs, (2) secretome from MSCs, or (3) exosomes produced by MSCs, wherein the MSCs overexpress TGFβ1, or (4) exosomes comprising one or more effectors, wherein the one or more effectors comprises TGFβ1.

13. The method of claim 1, wherein the method comprises administering the (1) MSCs, (2) secretome from MSCs, or (3) exosomes produced by MSCs, wherein the MSCs overexpress IL-10, or (4) exosomes comprising one or more effectors, wherein the one or more effectors comprises IL-10.

14. The method of claim 4, wherein the number of exosomes administered is from about 6E10 to about 6E12.

15. The method of claim 1, wherein the MSCs are placenta, bone marrow, or umbilical cord MSCs.

16. (canceled)

17. The method of claim 1, wherein the method is a method of treating or preventing POI.

18. The method of claim 17, wherein the POI is chemotherapy-induced.

19. (canceled)

20. The method of claim 1, wherein the method is a method of treating or preventing PCOS.

21.-22. (canceled)

23. A method of preparing mesenchymal stem cells (MSCs) for treating or preventing premature ovarian insufficiency (POI) or polycystic ovary syndrome (PCOS) in a female mammal, the method comprising collecting MSCs in an amount of about 4×10⁷ for administration in a single dosage.

24. The method of claim 1, wherein the female mammal is human.

25. A method of preparing ovarian tissue-specific exosomes and/or secretome, the method comprising co-culturing human ovarian granulosa cells (hGrC1) with mesenchymal stem cells (MSCs).

26. A method of treating or preventing premature ovarian insufficiency (POI) or polycystic ovary syndrome (PCOS) in a female mammal, the method comprising administering to the female mammal an effective amount of exosomes or secretome prepared according to claim 25.

27. The method of claim 26, wherein the female mammal is human.

28. A method of preventing chemotherapy-induced damage in a male mammal, the method comprising administering to the mammal an effective amount of exosomes and/or secretome prepared from mesenchymal stem cells (MSCs).

29. The method of claim 28, wherein the male mammal is a human.


SEQUENCE LISTING (57 sequences)

SEQ ID NO: 1 - RNA, 22 nt, Homo sapiens
  source 1..22: mol_type = other RNA
ggatatcatcatatactgtaag

SEQ ID NO: 2 - DNA, 22 bp, synthetic construct
  source 1..22: mol_type = other DNA
tggatcagcaagcaggagtatg

SEQ ID NO: 3 - DNA, 18 bp, synthetic construct
  source 1..18: mol_type = other DNA
gcatttgcggtggacgat

SEQ ID NO: 4 - DNA, 22 bp, synthetic construct
  source 1..22: mol_type = other DNA
ggcctcaaatggcaactctaga

SEQ ID NO: 5 - DNA, 21 bp, synthetic construct
  source 1..21: mol_type = other DNA
cttctgatcgccatccttgaa

SEQ ID NO: 6 - DNA, 18 bp, synthetic construct
  source 1..18: mol_type = other DNA
gagggagacgggcacaca

SEQ ID NO: 7 - DNA, 22 bp, synthetic construct
  source 1..22: mol_type = other DNA
tgacataaaccgactccacgtt

SEQ ID NO: 8 - DNA, 21 bp, synthetic construct
  source 1..21: mol_type = other DNA
caattcccggaggactacagt

SEQ ID NO: 9 - DNA, 21 bp, synthetic construct
  source 1..21: mol_type = other DNA
agcacgaatgtgaagttctgg

SEQ ID NO: 10 - DNA, 21 bp, synthetic construct
  source 1..21: mol_type = other DNA
tgcactttatgacgcactcac

SEQ ID NO: 11 - DNA, 23 bp, synthetic construct
  source 1..23: mol_type = other DNA
tgtccaaaaacacgaaatcatgc

SEQ ID NO: 12 - DNA, 23 bp, synthetic construct
  source 1..23: mol_type = other DNA
atgatggcttattacagtggcaa

SEQ ID NO: 13 - DNA, 20 bp, synthetic construct
  source 1..20: mol_type = other DNA
gtcggagattcgtagctgga

SEQ ID NO: 14 - DNA, 21 bp, synthetic construct
  source 1..21: mol_type = other DNA
aagcacactggtttccacact

SEQ ID NO: 15 - DNA, 20 bp, synthetic construct
  source 1..20: mol_type = other DNA
tgggtccctgcatatccgtt

SEQ ID NO: 16 - DNA, 20 bp, synthetic construct
  source 1..20: mol_type = other DNA
cacattgggggtaggaacac

SEQ ID NO: 17 - DNA, 20 bp, synthetic construct
  source 1..20: mol_type = other DNA
aactttggcattgtggaagg

SEQ ID NO: 18 - DNA, 20 bp, synthetic construct
  source 1..20: mol_type = other DNA
gagtttgccatcccgaagga

SEQ ID NO: 19 - DNA, 20 bp, synthetic construct
  source 1..20: mol_type = other DNA
ccagctccgaagggcaaata

SEQ ID NO: 20 - DNA, 20 bp, synthetic construct
  source 1..20: mol_type = other DNA
tttcgctgagagacgtggag

SEQ ID NO: 21 - DNA, 20 bp, synthetic construct
  source 1..20: mol_type = other DNA
aggattgctgcttcgacctc

SEQ ID NO: 22 - DNA, 20 bp, synthetic construct
  source 1..20: mol_type = other DNA
gtgcattcaacggaacccag

SEQ ID NO: 23 - DNA, 20 bp, synthetic construct
  source 1..20: mol_type = other DNA
tctaagccatggttgggcag

SEQ ID NO: 24 - DNA, 20 bp, synthetic construct
  source 1..20: mol_type = other DNA
gtacctccaccatgcccagt

SEQ ID NO: 25 - DNA, 20 bp, synthetic construct
  source 1..20: mol_type = other DNA
gcattcacatctgctgtgct

SEQ ID NO: 26 - DNA, 21 bp, synthetic construct
  source 1..21: mol_type = other DNA
ttcaggcaggcagtatcactc

SEQ ID NO: 27 - DNA, 21 bp, synthetic construct
  source 1..21: mol_type = other DNA
gaaggtccacgggaaagacac

SEQ ID NO: 28 - DNA, 24 bp, synthetic construct
  source 1..24: mol_type = other DNA
atccagttgccttcttgggactga

SEQ ID NO: 29 - DNA, 24 bp, synthetic construct
  source 1..24: mol_type = other DNA
taagcctccgacttgtgaagtggt

SEQ ID NO: 30 - DNA, 22 bp, synthetic construct
  source 1..22: mol_type = other DNA
cgggaagacaataactgcaccc

SEQ ID NO: 31 - DNA, 23 bp, synthetic construct
  source 1..23: mol_type = other DNA
cggttagcagtatgttgtccagc

SEQ ID NO: 32 - DNA, 20 bp, synthetic construct
  source 1..20: mol_type = other DNA
agatgcagttaacgccccac

SEQ ID NO: 33 - DNA, 20 bp, synthetic construct
  source 1..20: mol_type = other DNA
acccattccttcttggggtc

SEQ ID NO: 34 - DNA, 24 bp, synthetic construct
  source 1..24: mol_type = other DNA
acgtcagtacaaggagatgttgga

SEQ ID NO: 35 - DNA, 26 bp, synthetic construct
  source 1..26: mol_type = other DNA
atcctattgcagaatgcttctttacc

SEQ ID NO: 36 - DNA, 20 bp, synthetic construct
  source 1..20: mol_type = other DNA
tcccaggttctcttcaaggg

SEQ ID NO: 37 - DNA, 20 bp, synthetic construct
  source 1..20: mol_type = other DNA
ggtgaggagcacgtagtcgg

SEQ ID NO: 38 - DNA, 20 bp, synthetic construct
  source 1..20: mol_type = other DNA
ctgcctgcgaagagagcatc

SEQ ID NO: 39 - DNA, 20 bp, synthetic construct
  source 1..20: mol_type = other DNA
agctccacttcgccttttgg

SEQ ID NO: 40 - DNA, 21 bp, synthetic construct
  source 1..21: mol_type = other DNA
ttcccggcagcaccagtaacc

SEQ ID NO: 41 - DNA, 21 bp, synthetic construct
  source 1..21: mol_type = other DNA
tccctctttgcgtttggacta

SEQ ID NO: 42 - DNA, 22 bp, synthetic construct
  source 1..22: mol_type = other DNA
ttgcaagagagctgcatcagtt

SEQ ID NO: 43 - DNA, 23 bp, synthetic construct
  source 1..23: mol_type = other DNA
actgtgtgtggaaatagatgggc

SEQ ID NO: 44 - DNA, 24 bp, synthetic construct
  source 1..24: mol_type = other DNA
gcctggctttgaagacataatccg

SEQ ID NO: 45 - DNA, 24 bp, synthetic construct
  source 1..24: mol_type = other DNA
tcttgatagtgacgcttctgggac

SEQ ID NO: 46 - DNA, 20 bp, synthetic construct
  source 1..20: mol_type = other DNA
ggcccatcatgctatggaac

SEQ ID NO: 47 - DNA, 20 bp, synthetic construct
  source 1..20: mol_type = other DNA
gtgagggatcactcgccatc

SEQ ID NO: 48 - DNA, 18 bp, synthetic construct
  source 1..18: mol_type = other DNA
ttgccatattcctcacca

SEQ ID NO: 49 - DNA, 21 bp, synthetic construct
  source 1..21: mol_type = other DNA
cccactcgatcagatgtgctc

SEQ ID NO: 50 - DNA, 23 bp, synthetic construct
  source 1..23: mol_type = other DNA
caaaaacagaaggattgccgaaa

SEQ ID NO: 51 - DNA, 21 bp, synthetic construct
  source 1..21: mol_type = other DNA
tcttggactgagtcgtagagg

SEQ ID NO: 52 - DNA, 21 bp, synthetic construct
  source 1..21: mol_type = other DNA
tatggagtgacatagagtgtg

SEQ ID NO: 53 - DNA, 21 bp, synthetic construct
  source 1..21: mol_type = other DNA
gtcgctacaccacttcaatcc

SEQ ID NO: 54 - DNA, 22 bp, synthetic construct
  source 1..22: mol_type = other DNA
tgacattcatgggattgcagac

SEQ ID NO: 55 - DNA, 23 bp, synthetic construct
  source 1..23: mol_type = other DNA
catggtttgaaactcgaaaaggg

SEQ ID NO: 56 - DNA, 19 bp, synthetic construct
  source 1..19: mol_type = other DNA
ccaccagcgaggacttcac

SEQ ID NO: 57 - DNA, 21 bp, synthetic construct
  source 1..21: mol_type = other DNA
ggaggactctcgtagctcgaa